(19) United States
(12) Patent Application Publication
(10) Pub. No.: US 2021/0251276 A1
(43) Pub. Date: Aug. 19, 2021

(54) LAYERED FLEECE FOR POUCHED PRODUCT

(71) Applicant: NICOVENTURES TRADING LIMITED, London (GB)

(72) Inventors: Savannah Johnson, Winston-Salem, NC (US); Dwayne William Beeson, Kernersville, NC (US); Ronald K. Hutchens, East Bend, NC (US); Wesley Steven Jones, Lexington, NC (US); David Neil McClanahan, Winston-Salem, NC (US); Travis O'Neal, Pinnacle, NC (US); Pankaj Patel, Clemmons, NC (US)

(21) Appl. No.: 17/173,799

(22) Filed: Feb. 11, 2021

Related U.S. Application Data
(63) Continuation of application No. PCT/IB2020/061602, filed on Dec. 7, 2020.
(60) Provisional application No. 62/945,473, filed on Dec. 9, 2019.

(51) Int. Cl. A24B 13/00; A24B 15/16; B32B 5/06; B32B 7/022; B32B 5/02; B32B 5/26; A61K 9/00; A61K 31/465; A61K 45/06

(52) U.S. Cl. CPC A24B 13/00; A24B 15/16; B32B 5/06; B32B 7/022; B32B 5/022; B32B 2250/20; A61K 9/009; A61K 31/465; A61K 45/06; B32B 2307/726; B32B 5/265

(57) ABSTRACT

The disclosure provides oral pouched products in the form of a porous pouch defining a cavity containing a composition adapted for oral use, wherein the porous pouch is formed from a fleece material comprising two or more layers, wherein the two or more layers are in direct contact with one another. The disclosure also provides pouched products that include at least one layer of fleece material and at least one layer of a gel that is in direct contact with the fleece material. The disclosure also provides methods of forming such pouched products and for controlling/tailoring certain properties of pouched products using such multi-layered and/or gel-based approaches.

### FIELD OF THE DISCLOSURE

The present disclosure relates to pouched products intended for human use. The pouched products are configured for oral use and deliver substances such as flavors and/or active ingredients during use. Such products may include tobacco or a product derived from tobacco, or may be tobacco-free alternatives.

### BACKGROUND

Tobacco may be enjoyed in a so-called “smokeless” form. Particularly popular smokeless tobacco products are employed by inserting some form of processed tobacco or tobacco-containing formulation into the mouth of the user. Conventional formats for such smokeless tobacco products include moist snuff, snus, and chewing tobacco, which are typically formed almost entirely of particulate, granular, or shredded tobacco, and which are either portioned by the user or presented to the user in individual portions, such as in single-use pouches or sachets. Other traditional forms of smokeless products include compressed or agglomerated forms, such as plugs, tablets, or pellets. Alternative product formats, such as tobacco-containing gums and mixtures of tobacco with other plant materials, are also known.

See for example, the types of smokeless tobacco formulations, ingredients, and processing methodologies set forth in U.S. Pat. No. 1,376,586 to Schwartz; U.S. Pat. No. 4,513,756 to Pittman et al.; U.S. Pat. No. 4,528,993 to Sensabaugh, Jr. et al.; U.S. Pat. No. 4,624,269 to Story et al.; U.S. Pat. No. 4,991,599 to Tibbetts; U.S. Pat. No. 4,987,907 to Townsend; U.S. Pat. No. 5,092,352 to Sprinkle, III et al.; U.S. Pat. No. 5,387,416 to White et al.; U.S. Pat. No. 6,668,839 to Williams; U.S. Pat. No. 6,834,654 to Williams; U.S. Pat. No. 6,953,040 to Atchley et al.; U.S. Pat. No. 7,032,601 to Atchley et al.; and U.S. Pat. No. 7,694,686 to Atchley et al.; US Pat. Pub. Nos. 2004/0020503 to Williams; 2005/0115580 to Quinter et al.; 2006/0191548 to Strickland et al.; 2007/0062549 to Holton, Jr. et al.; 2007/0186941 to Holton, Jr. et al.; 2007/0186942 to Strickland et al.; 2008/0029110 to Dube et al.; 2008/0029116 to Robinson et al.; 2008/0173317 to Robinson et al.; 2008/0209586 to Neilsen et al.; 2009/0065013 to Essen et al.; and 2010/0282267 to Atchley, as well as WO2004/095959 to Arnarp et al., each of which is incorporated herein by reference.

Smokeless tobacco product configurations that combine tobacco material with various binders and fillers have been proposed more recently, with example product formats including lozenges, pastilles, gels, extruded forms, and the like. See, for example, the types of products described in US Patent App. Pub. Nos. 2008/0196730 to Engstrom et al.; 2008/0305216 to Crawford et al.; 2009/0293889 to Kumar et al.; 2010/0291245 to Gao et al; 2011/0139164 to Mua et al.; 2012/0037175 to Cantrell et al.; 2012/0055494 to Hunt et al.; 2012/0138073 to Cantrell et al.; 2012/0138074 to Cantrell et al.; 2013/0074855 to Holton, Jr.; 2013/0074856 to Holton, Jr.; 2013/0152953 to Mua et al.; 2013/0274296 to Jackson et al.; 2015/0068545 to Moldoveanu et al.; 2015/0101627 to Marshall et al.; and 2015/0230515 to Lampe et al., each of which is incorporated herein by reference.

All-white snus portions are growing in popularity, and offer a discrete and aesthetically pleasing alternative to traditional snus. Such modern “white” pouched products may include a bleached tobacco or may be tobacco-free.

### BRIEF SUMMARY

The present disclosure generally provides pouched products comprising a composition adapted for oral use within a porous pouch. In some embodiments, the porous pouch comprises a fleece material comprising two or more layers. In some embodiments, the porous pouch comprises at least one layer of fleece material and at least one layer of a gel.

In one aspect, the disclosure provides an oral pouched product in the form of a porous pouch defining a cavity containing a composition adapted for oral use, wherein the porous pouch is formed from a fleece material comprising two or more layers, wherein the two or more layers are in direct contact with one another. The fleece material comprising two or more layers may, in some embodiments, completely enclose the cavity. In some embodiments, the two or more layers are laminated to one another. In some embodiments, the fleece material includes three or more layers, four or more layers, or five or more layers (each layer being the same or different than one another).

In certain embodiments, the two or more layers comprise an inner layer adjacent to the composition adapted for oral use and an outer layer forming an outer surface of the pouched product, wherein the inner layer and the outer layer each have different physical properties. In some embodiments, the physical properties are selected from the group consisting of softness, mouth feel, dissolution properties, stain resistance/blocking, and combinations thereof. In some embodiments, the outer layer is softer than the inner layer.

In certain embodiments, at least one of the inner layer and the outer layer comprises an active ingredient, a flavorant, or both. For example, the outer layer can comprise an active ingredient, a flavorant, or both; and the outer layer can be configured to provide fast release of the active ingredient, the flavorant, or both, and the inner layer is configured for stain resistance. As another example, the outer layer can comprise a first active ingredient, a first flavorant, or both; and the inner layer can comprise a second active ingredient, a second flavorant, or both; wherein the first active ingredient and the first flavorant are different from the second active ingredient and the second flavorant.

The method by which such layered pouches are provided can vary. Generally, the disclosure provides a method of preparing pouched products, comprising: combining two or more nonwoven layers to give a fleece material and enclosing a composition adapted for oral use within a porous pouch formed from such fleece material. In some embodiments, a laminated structure is prepared using needle punching, and the laminated structure is subsequently extruded into the fleece material. In some embodiments, two or more active ingredients are layered on the laminated structure prior to needle punching, and wherein the two or more active ingredients are distributed throughout the resulting pouched product.

In another aspect, the disclosure provides an oral pouched product comprising a composition adapted for oral use within a porous pouch, the porous pouch comprising at least one layer of fleece material and at least one layer of a gel; wherein the gel is in direct contact with the at least one layer of fleece material. In some embodiments, the porous pouch comprises an inner surface and an outer surface, and wherein the at least one layer of gel is adhered to the inner surface of the porous pouch. In some embodiments, the porous pouch comprises an inner surface and an outer surface, and wherein the at least one layer of gel is adhered to the outer surface of the porous pouch. It is also envisioned that, in some embodiments, a layer of gel can be positioned both on the inner and outer surfaces of a porous pouch.

In some embodiments, the gel comprises an active ingredient, a flavorant, or both, and wherein the gel is configured for rapid release or gradual release of the active ingredient, the flavorant, or both. In some embodiments, the gel comprises one or more flavorants, providing for substantially immediate release of flavor when the pouched product is placed in a user's mouth.

In another aspect, the disclosure provides a method of providing an oral product with a customized flavor release profile, comprising: selecting a first nonwoven material based on its release or barrier properties; selecting a second nonwoven material based on its release or barrier properties; incorporating an active ingredient and/or flavorant within one or both of the first and second nonwoven materials; constructing a multi-layered fleece material from the first and second nonwoven materials; forming a porous pouch from the multi-layered fleece material; and enclosing a composition adapted for oral use within the porous pouch. In some embodiments, the release properties are correlated with one or more of composition, basis weight, thickness, porosity, and pore size.

In a further aspect, the disclosure provides a method of preventing discoloration of an oral pouched product, comprising: selecting a first nonwoven material suitable to prevent passage of color associated with a composition adapted for oral use; selecting a second nonwoven material; constructing a multi-layered fleece material from the first and second nonwoven materials; forming a porous pouch from the multi-layered fleece material and enclosing the composition adapted for oral use within the porous pouch, such that the first nonwoven material is in direct contact with the composition adapted for oral use.

In certain embodiments of the foregoing oral pouched products and methods, the active ingredient is selected from the group consisting of a botanical material, a stimulant, an amino acid, a vitamin, an antioxidant, nicotine components, a nutraceutical, a cannabinoid, a cannabimimetic, a terpene, a pharmaceutical agent, and combinations thereof. In certain embodiments of the foregoing pouched products and methods, the one or more flavoring agents are selected from the group consisting of aldehydes, ketones, esters, terpenes, terpenoids, trigeminal sensates, and combinations thereof.

The disclosure includes, without limitations, the following embodiments.

Embodiment 1: An oral pouched product, comprising a composition adapted for oral use within a porous pouch, wherein the porous pouch comprises a fleece material having two or more layers, wherein the two or more layers are in direct contact with one another.

Embodiment 2: The oral pouched product of Embodiment 1, wherein the two or more layers are laminated to one another.

Embodiment 3: The oral pouched product of any of Embodiments 1 or 2, wherein the two or more layers include three or more layers, four or more layers, five or more layers, or six or more layers.

Embodiment 4: The oral pouched product of any of Embodiments 1-3, wherein the two or more layers comprise an inner layer adjacent to the composition adapted for oral use and an outer layer on the surface of the pouched product, wherein the inner layer and the outer layer each have different physical properties.

Embodiment 5: The oral pouched product of any of Embodiments 1-4, wherein the physical properties are selected from the group consisting of softness, mouth feel, dissolution properties, stain resistance, and combinations thereof.

Embodiment 6: The oral pouched product of any of Embodiments 1-5, wherein the outer layer is softer than the inner layer.

Embodiment 7: The oral pouched product of any of Embodiments 1-6, wherein at least one of the inner layer and the outer layer comprises an active ingredient, a flavorant, or both.

Embodiment 8: The oral pouched product of any of Embodiments 1-7, wherein the outer layer comprises an active ingredient, a flavorant, or both; and wherein the outer layer is configured to provide fast release of the active ingredient, the flavorant, or both, and the inner layer is configured for stain resistance.

Embodiment 9: The oral pouched product of any of Embodiments 1-8, wherein the outer layer comprises a first active ingredient, a first flavorant, or both; wherein the inner layer comprises a second active ingredient, a second flavorant, or both; and wherein the first active ingredient and the first flavorant are different from the second active ingredient and the second flavorant.

Embodiment 10: The oral pouched product of any of Embodiments 1-9, wherein the two or more layers are laminated to one another and the laminated structure is prepared using needle punching, and the laminated structure is subsequently extruded.

Embodiment 11: The oral pouched product of any of Embodiments 1-10, wherein two or more active ingredients are layered on the laminated structure prior to needle punching, and wherein the two or more active ingredients are distributed throughout the resulting pouched product.

Embodiment 12: A method of preparing the oral pouched product of any of Embodiments 1-11, comprising: combining the two or more layers to give the fleece material and enclosing the composition adapted for oral use within a porous pouch formed from such fleece material.

Embodiment 13: An oral pouched product comprising a composition adapted for oral use within a porous pouch, the porous pouch comprising at least one layer of fleece material and at least one layer of a gel; wherein the gel is in direct contact with the at least one layer of fleece material.

Embodiment 14: The oral pouched product of Embodiment 13, wherein the porous pouch comprises an inner surface and an outer surface, and wherein the at least one layer of gel is adhered to the inner surface of the porous pouch.

Embodiment 15: The oral pouched product of any of Embodiments 13-14, wherein the porous pouch comprises an inner surface and an outer surface, and wherein the at least one layer of gel is adhered to the outer surface of the porous pouch.

Embodiment 16: The oral pouched product of any of Embodiments 13-15, wherein the gel comprises an active ingredient, a flavorant, or both, and wherein the gel is configured for rapid release or gradual release of the active ingredient, the flavorant, or both.

Embodiment 17: The oral pouched product of any of Embodiments 13-15, wherein the gel comprises one or more flavorants, providing for substantially immediate release of flavor when the pouched product is placed in a user's mouth.

Embodiment 18: The oral pouched product of any of Embodiments 1-11 or 13-17, wherein the composition adapted for oral use comprises one or more active ingredients and/or flavorants.

Embodiment 19: The oral pouched product of Embodiment 18, wherein the active ingredient is selected from the group consisting of a botanical material, a stimulant, an amino acid, a vitamin, an antioxidant, nicotine components, a nutraceutical, a cannabinoid, a cannabimimetic, a terpene a pharmaceutical agent, and combinations thereof.

Embodiment 20: The oral pouched product of any of Embodiments 1-19, wherein the composition adapted for oral use comprises one or more salts, one or more sweeteners, one or more binding agents, one or more humectants, one or more gums, a tobacco material, or combinations thereof.

Embodiment 21: The oral pouched product of any of Embodiments 1-20, wherein the composition adapted for oral use is substantially free of a tobacco material, excluding any nicotine component present.

Embodiment 22: The oral pouched product of any of Embodiments 1-21, wherein the composition adapted for oral use comprises no more than about 10% by weight of a tobacco material, excluding any nicotine component present.

Embodiment 23: A method for providing tailored release of an active ingredient and/or flavorant, comprising appropriately selecting components for and/or adapting the oral pouched product of any of Embodiments 1-22.

Embodiment 24: A method of providing a customized flavor release profile, comprising: selecting a first nonwoven material based on its release or barrier properties; selecting a second nonwoven material based on its release or barrier properties; incorporating an active ingredient and/or flavorant within one or both of the first and second nonwoven materials; constructing a multi-layered fleece material from the first and second nonwoven materials; and forming a porous pouch from the multi-layered fleece material; and enclosing a composition adapted for oral use within the porous pouch.

Embodiment 25: The method of Embodiment 24, wherein the release properties are correlated with one or more of composition, basis weight, thickness, porosity, and pore size.

Embodiment 26: A method of preventing discoloration of an oral pouched product, comprising: selecting a first nonwoven material suitable to prevent passage of color associated with a composition adapted for oral use; selecting a second nonwoven material; constructing a multi-layered fleece material from the first and second nonwoven materials; forming a porous pouch from the multi-layered fleece material; and enclosing the material intended for oral use within the porous pouch, such that the first nonwoven material is in direct contact with the composition adapted for oral use.

Embodiment 27: Use of a laminate fleece material to prepare an oral pouched product, wherein the laminate fleece material comprises two or more layers, wherein the two or more layers are in direct contact with one another.

Embodiment 28: The use of Embodiment 27, wherein the pouched product comprises the laminate fleece material formed into a pouched product with a composition adapted for oral use therein.

Embodiment 29: Use of a laminate fleece material to prepare an oral pouched product with little to no discoloration from a colored composition adapted for oral use on the interior thereof, wherein the laminate fleece material comprises a first layer in contact with the colored composition, comprising a nonwoven material suitable to prevent passage of color from the colored composition, and a second layer that forms an exterior surface of the pouched product.

Embodiment 30: Use of a gel to prepare an oral pouched product, wherein the gel is provided on at least one layer of fleece material to give a gel-coated fleece material.

Embodiment 31: The use of Embodiment 30, wherein the oral pouched product comprises the gel-coated fleece material formed into a pouched product with a composition adapted for oral use therein.

Embodiment 32: The use of any of Embodiments 27-31, wherein the composition adapted for oral use comprises one or more active ingredients and/or flavorants.

Embodiment 33: The use of any of Embodiments 27-32, wherein the composition adapted for oral use is substantially free of a tobacco material, excluding any nicotine component present.

Embodiment 34: The oral pouched product, method, or use of any of Embodiments 1-33, wherein the gel comprises a gelling agent selected from the group consisting of alginic acid, bentonite, carbomer, carboxymethylcellulose calcium, carboxymethylcellulose sodium, carrageenan, colloidal silicon dioxide, ethyl cellulose, gelatin, gellan gum, guar gum, hydroxyethyl cellulose, hydroxyethylmethyl cellulose, hydroxypropyl cellulose, hydroxypropylmethyl cellulose, glyceryl behenate, glyceryl monooleate, magnesium aluminum silicate, methyl cellulose, poloxamer, polyethylene oxide, polyvinyl alcohol, povidone, propylene glycol alginate, sodium alginate, tragacanth, polyacrylic acid, and combinations thereof.

These and other features, aspects, and advantages of the disclosure will be apparent from a reading of the following detailed description together with the accompanying drawings, which are briefly described below. The invention includes any combination of two, three, four, or more of the above-noted embodiments as well as combinations of any two, three, four, or more features or elements set forth in this disclosure, regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise.

### BRIEF DESCRIPTION OF THE DRAWINGS

Having thus described aspects of the disclosure in the foregoing general terms, reference will now be made to the accompanying drawing, which is not necessarily drawn to scale. The drawing is exemplary only, and should not be construed as limiting the disclosure.

FIG. 1 is a perspective view of a pouched product according to an example embodiment of the present disclosure, including a pouch of fleece at least partially filled with a composition for oral use; and

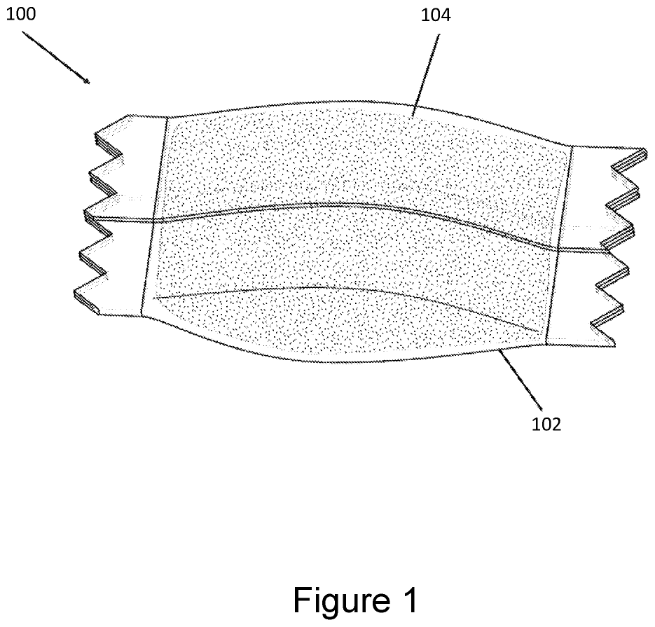

FIG. 2 is a schematic providing a cross-section of one embodiment of a multi-layered fleece material.

### DETAILED DESCRIPTION

The present disclosure provides a pouched product comprising a composition adapted for oral use within a porous pouch. In one aspect, the porous pouch comprises a fleece material having two or more layers, wherein the two or more layers are each in direct contact with one another. In another aspect, the porous pouch comprises at least one layer of fleece material and at least one layer of a gel, wherein the gel is in direct contact with the at least one layer of fleece material. The pouched products are configured to deliver active ingredients and/or flavorants to the consumer in an oral form.

The present disclosure will now be described more fully hereinafter with reference to example embodiments thereof. These example embodiments are described so that this disclosure will be thorough and complete, and will fully convey the scope of the disclosure to those skilled in the art. Indeed, the disclosure may be embodied in many different forms and should not be construed as limited to the embodiments set forth herein; rather, these embodiments are provided so that this disclosure will satisfy applicable legal requirements. As used in this specification and the claims, the singular forms “a,” “an,” and “the” include plural referents unless the context clearly dictates otherwise. Reference to “dry weight percent” or “dry weight basis” refers to weight on the basis of dry ingredients (i.e., all ingredients except water). Reference to “wet weight” refers to the weight of the mixture including water. Unless otherwise indicated, reference to “weight percent” of a mixture reflects the total wet weight of the mixture (i.e., including water). Reference to “substantially free” in regard to certain components means that the referenced component is not present, has not been intentionally added, and/or is present in only trace amounts in the composition. For example, less than 1%, less than 0.1%, less than 0.01%, less than 0.001%, or 0% of the referenced component may be present by weight in the composition.

The products as described herein are configured for oral use. The term “configured for oral use” as used herein means that the product is provided in a form such that during use, saliva in the mouth of the user causes one or more of the components of the product (e.g., flavoring agents and/or active ingredients) to pass into the mouth of the user. In certain embodiments, the product is adapted to deliver components to a user through mucous membranes in the user's mouth, the user's digestive system, or both, and, in some instances, said component is an active ingredient that can be absorbed through the mucous membranes in the mouth or absorbed through the digestive tract when the product is used.

The disclosure generally provides products in the form of a mixture of one or more components disposed within a moisture-permeable container (e.g., a water-permeable pouch). Such mixtures in the water-permeable pouch format are typically used by placing a pouch containing the mixture in the mouth of a human subject/user. Generally, the pouch is placed somewhere in the oral cavity of the user, for example under the lips, in the same way as moist snuff products are generally used. The pouch preferably is not chewed or swallowed. Exposure to saliva then causes some of the components of the mixture therein (e.g., flavoring agents and/or nicotine) to pass through e.g., the water-permeable pouch and provide the user with flavor and satisfaction, and the user is not required to spit out any portion of the mixture. After about 10 minutes to about 60 minutes, typically about 15 minutes to about 45 minutes, of use/enjoyment, substantial amounts of the mixture have been ingested by the human subject, and the pouch may be removed from the mouth of the consumer for disposal.

Certain embodiments of the disclosure will be described with reference to the figures of the accompanying drawings, and these described embodiments involve snus-type products having an outer pouch and containing a mixture of components (as referenced herein below). The pouched product 100 includes a moisture-permeable container in the form of a pouch 102, which contains a composition 104 comprising a mixture of components. As explained in greater detail below, such embodiments are provided by way of example only. In particular, the size and shape of the illustrated outer pouches can vary as described in detail herein. The mixture/construction of such packets or pouches, such as the container pouch 102 in the embodiment illustrated in the figures, may be varied.

Suitable materials for the packets, pouches or containers of the type used for the manufacture of smokeless tobacco products are available under the tradenames CatchDry, Ettan, General, Granit, Goteborgs Rape, Grovsnus White, Metropol Kaktus, Mocca Anis, Mocca Mint, Mocca Wintergreen, Kicks, Probe, Prince, Skruf and TreAnkrare. The mixture may be contained in pouches and packaged, in a manner and using the types of components used for the manufacture of conventional snus types of products. The pouch provides a liquid-permeable container of a type that may be considered to be similar in character to the mesh-like type of material that is used for the construction of a tea bag. Components of the mixture readily diffuse through the pouch and into the mouth of the user. Non-limiting examples of pouches are set forth in, for example, U.S. Pat. No. 5,167,244 to Kjerstad and U.S. Pat. No. 8,931,493 to Sebastian et al.; as well as US Patent App. Pub. Nos. 2016/0000140 to Sebastian et al.; 2016/0073689 to Sebastian et al.; 2016/0157515 to Chapman et al.; and 2016/0192703 to Sebastian et al., each of which are incorporated herein by reference. As provided herein, such example pouches are considered herein to be “conventional” products, which are provided as comparisons to the pouches disclosed herein, which exhibit various modifications with respect to one or more such conventional products. Pouches can be provided as individual pouches, or a plurality of pouches (e.g., 2, 4, 5, 10, 12, 15, 20, 25 or 30 pouches) can be connected or linked together (e.g., in an end-to-end manner) such that a single pouch or individual portion can be readily removed for use from a one-piece strand or matrix of pouches.

An example pouch may be manufactured from materials, and in such a manner, such that during use by the user, the pouch undergoes a controlled dispersion or dissolution. Such pouch materials may have the form of a mesh, screen, perforated paper, permeable fabric, or the like. For example, pouch material manufactured from a mesh-like form of rice paper, or perforated rice paper, may dissolve in the mouth of the user. As a result, the pouch and mixture each may undergo complete dispersion within the mouth of the user during normal conditions of use, and hence the pouch and mixture both may be ingested by the user. Other examples of pouch materials may be manufactured using water dispersible film forming materials (e.g., binding agents such as alginates, carboxymethylcellulose, xanthan gum, pullulan, and the like), as well as those materials in combination with materials such as ground cellulosics (e.g., fine particle size wood pulp). Preferred pouch materials, though water dispersible or dissolvable, may be designed and manufactured such that under conditions of normal use, a significant amount of the mixture contents permeate through the pouch material prior to the time that the pouch undergoes loss of its physical integrity. If desired, flavoring ingredients, disintegration aids, and other desired components, may be incorporated within, or applied to, the pouch material.

The disclosure, in particular, provides pouched products comprising a material within a porous pouch, wherein the porous pouch comprises a fleece material comprising two or more layers, as well as pouched products comprising a composition adapted for oral use within a porous pouch, wherein the porous pouch comprises at least one layer of fleece material and at least one layer of a gel. Each of these embodiments is described in further detail below, followed by an overview of common components that may, in various embodiments, be incorporated within any such pouched products.

### Porous Pouch Comprising Fleece Material Comprising Two or More Layers

In some embodiments, a pouched product is provided, which comprises a porous pouch comprising a material comprising two or more layers. The two or more layers can comprise the same or different materials. Examples of such materials include, but are not limited, to fleece materials. “Fleece materials,” as used herein, are understood to be materials generally formed from fibrous nonwoven webs (i.e., comprising fibers). As used herein, the term “fiber” is defined as a basic element of textiles. Fibers are often in the form of a rope- or string-like element. As used herein, the term “fiber” is intended to include fibers, filaments, continuous filaments, staple fibers, and the like. The term “multicomponent fibers” refers to fibers that comprise two or more components that are different by physical or chemical nature, including bicomponent fibers. Specifically, the term “multicomponent fibers” includes staple and continuous fibers prepared from two or more polymers present in discrete structured domains in the fiber, as opposed to blends where the domains tend to be dispersed, random or unstructured.

The term “nonwoven” is used herein in reference to fibrous materials, webs, mats, batts, or sheets in which fibers are aligned in an undefined or random orientation. In one embodiment, the nonwoven fibers are initially presented as continuous unbound fibers or filaments. The continuous fibers are aligned substantially parallel to one another in at least one direction. In certain embodiments, a first plurality of continuous fibers are aligned substantially parallel to each other in a first direction and a second plurality of continuous fibers are aligned substantially parallel to each other in a cross direction relative to the first plurality of continuous fibers. The manufacturing process for such nonwovens typically involves binding the various fibers or filaments together. The manner in which the fibers or filaments are bound can vary, and include thermal, mechanical and chemical techniques that are selected in part based on the desired characteristics of the final product. In some embodiments, the oriented fibers undergo a heat treatment process (e.g., a lamination process) in order to bind them together. Due to the defined orientation of the continuous fibers, the overlap of the individual fibers is low and a thin nonwoven fabric can be realized. The surface of the nonwoven fabric can also be uniform and smooth.

In some embodiments, the nonwoven fabric can be made in a spunlaid or spunmelt process, which includes both spunbond and meltblown processes, wherein such processes are understood to typically entail melting, extruding, collecting and bonding thermoplastic polymer materials to form a fibrous nonwoven web. Spunlaid nonwoven webs can be formed in a continuous process. Fibers can be spun and then directly dispersed into a web by deflectors or can be directed with air streams, for example. Spunbonding typically involves melt spinning, wherein a polymer is melted to a liquid state and forced through small orifices into cool air, such that the polymer strands solidify according to the shape of the orifices. The fiber bundles thus produced are then drawn, i.e., mechanically stretched (e.g., by a factor of 2-5) to orient the fibers. A nonwoven web is then formed by depositing the drawn fibers onto a moving belt. General spunbonding processes are described, for example, in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, and U.S. Pat. No. 3,542,615 to Dobo et al., which are all incorporated herein by reference.

Meltblowing is a process wherein a polymer (or polymers) is melted to a liquid state and extruded through a linear die containing numerous (e.g., several hundred or more) small orifices. As the polymer is extruded, streams of hot air are rapidly blown at the polymer, rapidly stretching and/or attenuating the extruded polymer streams to form extremely fine filaments. The air streams typically stretch or attenuate the molten polymer by many orders of magnitude. The stretched polymer fibers are collected as a randomly entangled, self-bonded nonwoven web. The technique of meltblowing is known in the art and is discussed in various patents, for example, U.S. Pat. No. 3,849,241 to Butin, U.S. Pat. No. 3,987,185 to Buntin et al., U.S. Pat. No. 3,972,759 to Buntin, and U.S. Pat. No. 4,622,259 to McAmish et al., each of which is herein incorporated by reference in its entirety.

In some embodiments, the nonwoven web can be made in a process that combines orientation and spinning technology. The nonwoven web can be formed by spinning continuous filament fibers, orienting the fibers parallel to one another in at least one direction, and bonding the fibers with heat. In various embodiments, the nonwoven web can be multi-layer where the multiple layers are laminated in more than one direction (e.g., cross-laminated). For example, continuous filaments can be aligned and layered along both MD and CD directions.

In heat bound embodiments, the nonwoven fabric (e.g., spunbond or meltblown web) can be formed using a thermoplastic polymer as a binder fiber. The thermoplastic polymer can exhibit a melting point in a relatively low range to facilitate heat sealing of the pouch material. For example, the thermoplastic polymer fiber can typically have a melting point of about 200° C. or less, about 160° C. or less, about 150° C. or less, about 140° C. or less, or about 120° C. or less. Example thermoplastic polymers include various polyolefin and polyester materials. For example, the binder fibers can comprise an aliphatic polyester. Advantageously, the thermoplastic polymer of the binder fibers can be a biodegradable polymer, such as an aliphatic polyester. Example aliphatic polyesters include polyglycolic acid (PGA), polylactic acid (PLA) (e.g., poly(L-lactic acid) or poly(DL-lactic acid)), polyhydroxyalkanoates (PHAs) such as polyhydroxypropionate, polyhydroxyvalerate, polyhydroxybutyrate, polyhydroxyhexanoate, and polyhydroxyoctanoate, polycaprolactone (PCL), polybutylene succinate, polybutylene succinate adipate, and copolymers thereof (e.g., polyhydroxybutyrate-co-hydroxyvalerate (PHBV)). Specific examples of commercially available PLA fibers include Ecodear® from Toray of Japan; Ingeo™ based PLA fibers from Fiber Innovations Technology, USA; and PLA fibers from Trevira GmbH. PLA and PHA materials can be sourced from a variety of plant materials, including tobacco.

In certain embodiments, the nonwoven fabric may include additional fiber types blended with the above-noted thermoplastic polymer fibers. Suitable fibers include those made of wool, cotton, regenerated cellulose, cellulose acetate, cellulose triacetate, cellulose nitrate, ethyl cellulose, cellulose acetate propionate, cellulose acetate butyrate, hydroxypropyl cellulose, methyl hydroxypropyl cellulose, protein fibers, and the like. See also, the fiber types set forth in US Pat. Appl. Pub. No. 2014/0083438 to Sebastian et al., which is incorporated by reference herein. Regenerated cellulose fibers are typically prepared by extracting non-cellulosic compounds from wood, contacting the extracted wood with caustic soda, followed by carbon disulfide and then by sodium hydroxide, giving a viscous solution. The solution is subsequently forced through spinneret heads to create viscous threads of regenerated fibers. Exemplary methods for the preparation of regenerated cellulose are provided in U.S. Pat. No. 4,237,274 to Leoni et al; U.S. Pat. No. 4,268,666 to Baldini et al; U.S. Pat. No. 4,252,766 to Baldini et al.; U.S. Pat. No. 4,388,256 to Ishida et al.; U.S. Pat. No. 4,535,028 to Yokogi et al.; U.S. Pat. No. 5,441,689 to Laity; U.S. Pat. No. 5,997,790 to Vos et al.; and U.S. Pat. No. 8,177,938 to Sumnicht, which are incorporated herein by reference. The manner in which the regenerated cellulose is made is not limiting, and can include, for example, both the rayon and the TENCEL processes. Various suppliers of regenerated cellulose are known, including Lenzing (Austria), Cordenka (Germany), Aditya Birla (India), and Daicel (Japan).

The fibers used in the nonwoven fabric according to the present disclosure can vary, and include fibers having any type of cross-section including, but not limited to, circular, rectangular, square, oval, triangular, and multilobal. In certain embodiments, the fibers can have one or more void spaces, wherein the void spaces can have, for example, circular, rectangular, square, oval, triangular, or multilobal cross-sections.

The physical parameters of the fibers present in the nonwoven fabric can vary. For example the fibers used in the nonwoven fabric can have varying size (e.g., length, dpf) and crimp characteristics. In some embodiments, fibers used in the nonwoven fabric can be nano fibers, sub-micron fibers, and/or micron-sized fibers. In certain embodiments, fibers useful herein can measure about 1.5 dpf to about 2.0 dpf, or about 1.6 dpf to about 1.90 dpf. In various embodiments, each fiber can measure about 4-10 crimps per cm, or about 5-8 crimps per cm. The fibers can be in staple form in certain embodiments, but advantageously, the fibers of the nonwoven fabric are in the form of continuous filaments.

The means of producing the nonwoven fabric can vary. Web formation can be accomplished by any means known in the art. As mentioned above, in various embodiments of the present disclosure, the nonwoven web can be produced by a spunbond process and or a meltblown process.

Nonwoven webs can have varying thicknesses, porosities and other parameters. The nonwoven web can be formed such that the fiber orientation and porosity of the pouched product formed therefrom can retain the composition that in some embodiments is enclosed within the pouch, but can also allow the components (e.g., active ingredient and flavor) of the composition to be enjoyed by the consumer. For example, the spunmelt nonwoven fabric can have a basis weight of about 18 gsm to about 80 gsm, or about 20 gsm to about 60 gsm, or about 22 gsm to about 30 gsm, for example. Basis weight of a fabric can be measured using ASTM D3776/D3776M-09a (2013) (Standard Test Methods for Mass Per Unit Area (Weight) of Fabric), for example. In various embodiments, the spunmelt nonwoven fabric can have a thickness of about 120 microns to about 300 microns, or about 130 microns to about 200 microns. In a preferred embodiment, the spunmelt nonwoven fabric can have a thickness of about 160 microns, for example. The spunmelt nonwoven fabric can have a dry tensile (machine direction) strength of about 750 N/m to about 950 N/m, or about 825 N/m to about 875 N/m, for example. The spunmelt nonwoven fabric can have a dry tensile (cross direction) strength of about 200 N/m to about 300 N/m, or about 220 N/m to about 260 N/m, for example. Dry tensile strength can be measured by, for example, ISO 1924-2:2008 (Paper and board—Determination of tensile properties—Part 2: Constant rate of elongation method (20 mm/min)). The spunmelt nonwoven fabric can have a dry elongation (machine direction) of about 8% to about 20%, or about 10% to about 16%, for example. The spunmelt nonwoven fabric can have a dry elongation (cross direction) of about 10% to about 20%, or about 14% to about 18%, for example. Elongation and breaking strength of textile fabrics can be measured using ASTM D5034-09 (2013) (Standard Test Method for Breaking Strength and Elongation of Textile Fabrics (Grab Test)), for example.

In some embodiments, the nonwoven fabric comprises nanocellulose. In some embodiments, the nanocellulose in the fleece material comprises cellulose nanofibrils (CNF) having a diameter and length, wherein the diameter is from about 1 to about 100 nm, and the length is from about 1 to about 10 micrometers. The quantity of nanocellulose present in the fleece material may vary. In some embodiments, the fleece material comprises at least about 10% by weight of the nanocellulose. For example, the fleece material may comprise from about 10%, about 20%, about 30%, about 40%, or about 50%, to about 60%, about 70%, about 80%, about 90%, about 95%, or about 99% by weight of the nanocellulose, based on the total weight of the fleece material.

Advantageously, by combining two or more layers of fleece material in certain embodiments, various beneficial properties can be exhibited by the resulting pouched product. The two or more layers can comprise, e.g., three or more layers, four or more layers, five or more layers, or even higher numbers of layers. The layered structure of the disclosed pouches can be provided, e.g., by independently providing two fleeces (e.g., two non-woven fabrics) and combining them such that the two fleeces are in contact with one another.

In some embodiments, the multi-layered fleece is provided by lamination of the two or more layers, i.e., the pouched product comprises a pouch comprising a laminated multi-layer material. Lamination generally involves the bonding of two or more layers by the application of heat and pressure. One of skill in the art would recognize the requisite temperature and pressure required to produce a suitable laminate of any two or more nonwoven layers as provided herein to produce a multilayer pouch material.

In some embodiments, the multi-layered fleece is provided by attaching the two or more fleece layers together using adhesive or stitching. Where the multi-layered fleece comprises a wet-laid material, multiple headboxes could be used to attach the layers together. In some embodiments, the multi-layered fleece is provided by needle punching of the two or more layers. In certain embodiments, a laminated structure is prepared using needle punching, and the laminated structure is subsequently extruded. Active ingredients and/or flavorants can, in some embodiments, be layered on the laminated structure prior to needle punching, and the active ingredient(s) and/or flavorant(s) are distributed throughout the resulting pouched product.

In such embodiments, therefore, there may be some penetration of a component in one layer into another layer via such processing. For example, where discrete layers are provided, one layer comprising a first flavorant and a second layer comprising a second flavorant, the process of needle punching may result in some migration of the first flavorant to the second layer and/or the second flavorant to the first layer.

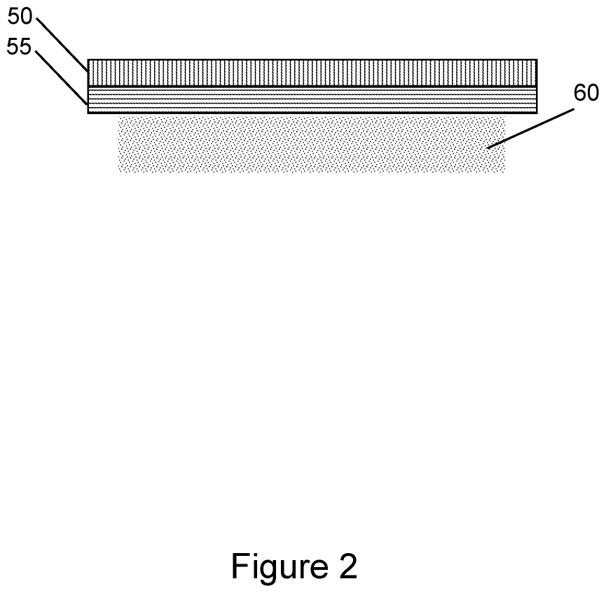

A representative schematic of a two-layered fleece is provided as FIG. 2. The two-layered fleece shown comprises an outer layer 50 and an inner layer 55 that can be in contact with the composition adapted for oral use 60. As such, typically only one layer is in contact with the composition adapted for oral use and typically only one later forms the outer surface of the pouched product. Of course, where the multi-layered fleece comprises more than two layers, it follows that at least one layer is not on the inner or outer surfaces (i.e., it is not directly exposed to the composition for oral use and is not directly exposed to the outer atmosphere). As such, in some embodiments, the multi-layered configurations allows for the use of layers that may otherwise be unsuitable for use within a pouched product (e.g., a layer that should not be in direct contact with a composition adapted for oral use can be positioned as the outer layer 50 or in a further layer between layers 50 and 55 and a layer that should not be in contact with the environment/mouth of the user can be positioned as the inner layer 55 or in a further layer between layers 50 and 55).

Although the pouch materials described with respect to such “multi-layer” embodiments are described as comprising two or more “layers,” it is understood that, due to processing, etc., the two or more layers may not always be present in discrete layers; i.e., there may be some overlap or permeation from one layer into the next. Such materials are still intended to be encompassed within the disclosure of “multi-layered” fleece-containing materials as provided herein. In other embodiments, overlap/permeation between two layers is advantageously avoided. For example, in some embodiments, flavor within an outer layer is beneficially first released before release of a flavor within an inner layer and, in certain embodiments, such flavorants can be segregated by the inclusion of a further layer therebetween. For example, in some such embodiments, a “barrier” layer may be included between the two flavorant-containing layers, thus preventing the different flavors from blending. The release of flavorants can, in some embodiments, be controlled via modifying the hydrophobicity of the layers, e.g., such that a hydrophobic layer can prevent moisture from leaving the inner composition until enough moisture from the mouth of the user overwhelms the hydrophobic layer and thereby allows moisture to enter and leave the inner area of the pouched product where the composition is housed.

The two or more layers of the pouches disclosed herein may comprise layers that are the same or different, or a combination thereof (e.g., comprising two identical layers and one layer that differs, as described herein below).

In some embodiments, products are provided which comprise two or more identical fleece layers. Such fleece layers can be, for example, provided without any additional components or can be treated with one or more additional components. For example, in some embodiments, the layers can comprise a flavor component (such as any of the flavor components noted herein), which can be applied to the nonwoven layer in any conventional manner such as by coating, printing, and the like. In some embodiments, the fleece layers can comprise an active component (e.g., including, but not nicotine, as outlined herein below).

In some embodiments, products are provided which comprise two or more fleece layers, wherein the fleece layers themselves are different. For example, two layers may be the same compositionally (e.g., comprised of the same types of fibers), but differ in physical properties (e.g., including, but not limited to, porosity, basis weight, thickness, and the like). As another example, two layers may differ compositionally (e.g., comprising different types of fibers within the nonwoven sheet). In some embodiments, the two or more fleece layers may differ both in terms of composition and physical properties.

In some embodiments, products are provided which comprise two or more layers of the same fleece material, with different components associated therewith (e.g., with different distributions and/or types of the “active components” referenced herein below). Such an embodiment may allow, e.g., for inclusion of different amounts of the same components in various layers, e.g., providing for varying release profiles of the same component during use of the corresponding pouched product. Such an embodiment may also allow, e.g., for inclusion of different types of components in different layers (e.g., different flavorants). In some embodiments, the physical properties of the individual layers of fleece material can afford some level of control of release of components therefrom, e.g., allowing for extended release from one layer (e.g., having lower porosity and/or higher thickness).

In certain embodiments, one of the two or more fleece layers can be relatively hydrophilic and one of two or more fleece layers can be relatively hydrophobic. The relatively hydrophobic layer can be positioned between the composition within the cavity of the pouch and the relatively hydrophilic layer, for example. In certain embodiments, the relatively hydrophilic layer can comprise a flavor component, as referenced, e.g., in U.S. Patent Application Publication No. 2016/0192703 to Sebastian et al., which is incorporated herein by reference in its entirety.

In certain embodiments, one of the two or more fleece layers can be specifically designed so as to counteract discoloration of the outer layer. For example, where the composition adapted for oral use 60 is colored, inner layer 55 may advantageously be comprised of a material designed to prevent such color from permeating through to outer layer 50 (maintaining, e.g., a white color associated with outer layer 50). One of skill in the art would recognize methods for tailoring inner layer 55 to prevent permeation of color from the composition 60 therethrough (e.g., by controlling porosity, pore size, or other physical features of the fleece layer, by selecting a particular material for construction of the fleece, etc.). Accordingly, a method of reducing discoloration of the outer pouch material of a pouched product is provided herein, via the use of such a layered construction.

In certain embodiments, the multi-layered design provided herein can allow for the use of a material as outer layer 50 that has improved mouthfeel (e.g., including, but not limited to, increased softness). It is recognized that some materials that could provide for such positive sensory characteristics may not be particularly advantageous to serve other functions necessary within a pouched product, e.g., for retaining composition 60, for stain resistance, for holding/releasing flavor and/or active components, and the like. By employing an outer material 50 provided solely for its sensory characteristics, and combining it with an inner material 55 designed to exhibit other beneficial characteristics (e.g., the types of functions that may not be adequately served by outer layer 50), a suitable material with improved mouthfeel is provided. Accordingly, a method of providing a pouched product with improved mouthfeel is provided herein, via the use of such a layered construction.

In certain embodiments, the multi-layered design provided herein can allow for the use of a material as outer layer 50 that has improved release properties (e.g., to allow for fast release of flavorants and/or active components) therefrom. Again, this functionality can be combined, via the disclosed multi-layer construction, with one or more other, interior layers with other types of functionality (e.g., prevention of discoloration, for holding/releasing flavor and/or active components later in use, etc.).

It is to be understood that, based on the foregoing, various combinations of properties can be provided by combining two or more layers are generally outlined herein. The benefits of various combinations are dependent upon the exact compositions and the exact functionalities of each constituent layer. One of skill in the art will appreciate, based on the foregoing, advantageous combinations of two, three, or more such layers, to obtain pouched products exhibiting a range of advantageous characteristics.

### Porous Pouch Comprising Fleece Material and Gel Layer

In some embodiments, a pouched product is provided, which comprises a porous pouch comprising a material comprising a first, nonwoven fleece material and a gel. A gel as used herein is understood to be a material that is a colloid (aggregate of fine particles, dispersed or arranged in a continuous medium). The properties of gels can range from being elastic and jelly-like to more rigid and solid. Typically in a gel, the continuous liquid medium has become viscous enough such that the gel behaves more or less like a solid. Methods of preparing gels, particularly for use in oral applications, are known (e.g., from the pharmaceutical arts).

Typically, gels comprise substantially dilute cross-linked polymers which exhibit no flow in the solid state. Some gels are hydrocolloids (i.e., gels comprising particles dispersed in water). Many gels exhibit reversibility between the gel state (e.g., at cooler temperatures) and the sol state (when heated). Other gels exhibit irreversibility between such states, e.g., as they may comprise polymer chains that are covalently bonded.

The types of gels employed herein are not particularly limited. Advantageously, the gels incorporated within the disclosed pouched products are inert, safe, and non-reactive with other components of the porous pouch (particularly non-reactive with other components in contact therewith). Gels can comprise just the polymer component and the solvent component, but may, in some embodiments, further comprise one or more stabilizers, penetration enhancers, and/or preservatives.

Certain gelling agents employed to produce gels that can be used in accordance with various embodiments of the present disclosure include, but are not limited to, alginic acid, bentonite, carbomer, carboxymethylcellulose calcium, carboxymethylcellulose sodium, carrageenan, colloidal silicon dioxide, ethyl cellulose, gelatin, gellan gum, guar gum, hydroxyethyl cellulose, hydroxyethylmethyl cellulose, hydroxypropyl cellulose, hydroxypropylmethyl cellulose, glyceryl behenate, glyceryl monooleate, magnesium aluminum silicate, methyl cellulose, poloxamer, polyethylene oxide, polyvinyl alcohol, povidone, propylene glycol alginate, sodium alginate, tragacanth, and polyacrylic acid. Such gelling agents can be combined, e.g., with water (other solvents include, but are not limited to, glycerin, glycerol, alcohols (e.g., ethanol), sucrose, toluene, mineral oils, etc.).

The gel layer provided herein can be provided alone or in combination with one or more other components. For example, in some embodiments, one or more flavorants and/or one or more active ingredients may be incorporated within a gel (e.g., dissolved or dispersed therein). Such components may, in some embodiments, simply be added during production of the gel in the desired amount.

Some gels provide for extended release of components associated therewith. In some embodiments, the rate of release can be modified to some extent by modifying the concentration of polymer:water. Some gels are themosensitive and may be employed to release components upon reaching a certain temperature (e.g., within the user's mouth). In particular embodiment, the gel is a hydrogel, which comprises a polymeric system formed through cross-linking which, when subjected to an aqueous environment, swells. Such materials are highly porous, which enhances their ability to hold one or more components (e.g., active ingredients and/or flavorants).

A gel as provided herein can be incorporated within a pouched product in various manners. In some embodiments, a gel is associated with a fleece layer as a coating thereon. The coating can be formed via, e.g., formation of the gel directly on the fleece layer, or by separate production of the gel and subsequent combination with the fleece layer.

As outlined with respect to the multi-layered fleeces described herein above, the combination of a non-woven layer and a gel layer can allow for tailoring of a pouched product into which it is incorporated. Numerous options are provided for incorporating one or more flavorants and/or one or more active ingredients within one or both of the non-woven layer and the gel layer. Further, the gel layer may be provided on the outside of the pouched product or on the inside of the pouched product (adjacent to the composition contained therein). As such, inclusion of a gel layer can afford, e.g., modification of flavor release profiles, variability in active ingredient release, and/or improved organoleptic sensation (e.g., gel-like coating that can be described as smooth, pasty, fluffy, or the like in use). As referenced above for multi-layer fleeces, in some embodiments, the gel may be provided as an interior layer to block discoloration of the outer nonwoven layer by serving as a barrier to permeation from color associated with the composition contained within the pouch.

### Features of the Multi-Layered and Gel-Containing Pouched Products Provided Herein

The multi-layered and gel-containing pouched products as described herein can be packaged within any suitable inner packaging material and/or outer container. See also, for example, the various types of containers for smokeless types of products that are set forth in U.S. Pat. No. 7,014,039 to Henson et al.; U.S. Pat. No. 7,537,110 to Kutsch et al.; U.S. Pat. No. 7,584,843 to Kutsch et al.; U.S. Pat. No. 8,397,945 to Gelardi et al., D592,956 to Thiellier; D594,154 to Patel et al.; and D625,178 to Bailey et al.; US Pat. Pub. Nos. 2008/0173317 to Robinson et al.; 2009/0014343 to Clark et al.; 2009/0014450 to Bjorkholm; 2009/0250360 to Bellamah et al.; 2009/0266837 to Gelardi et al.; 2009/0223989 to Gelardi; 2009/0230003 to Thiellier; 2010/0084424 to Gelardi; and 2010/0133140 to Bailey et al; 2010/0264157 to Bailey et al.; and 2011/0168712 to Bailey et al. which are incorporated herein by reference.

The composition within the pouched products of the present disclosure may be dissolvable. As used herein, the terms “dissolve,” “dissolving,” and “dissolvable” refer to compositions having aqueous-soluble components that interact with moisture in the oral cavity and enter into solution, thereby causing gradual consumption of the product. According to one aspect, the dissolvable product is capable of lasting in the user's mouth for a given period of time until it completely dissolves. Dissolution rates can vary over a wide range, from about 1 minute or less to about 60 minutes. For example, fast release compositions typically dissolve and/or release the active substance in about 2 minutes or less, often about 1 minute or less (e.g., about 50 seconds or less, about 40 seconds or less, about 30 seconds or less, or about 20 seconds or less). Dissolution can occur by any means, such as melting, mechanical disruption (e.g., chewing), enzymatic or other chemical degradation, or by disruption of the interaction between the components of the composition. In some embodiments, the product can be meltable as discussed, for example, in US Patent App. Pub. No. 2012/0037175 to Cantrell et al. In other embodiments, the products do not dissolve during the product's residence in the user's mouth.

The amount of composition contained within each pouch may vary. In some embodiments, the weight of the composition within each pouch is at least about 50 mg, for example, from about 50 mg to about 2 grams, from about 100 mg to about 1.5 grams, or from about 200 to about 700 mg. In some smaller embodiments, the weight of the composition within each pouch may be from about 100 to about 300 mg. For a larger embodiment, the weight of the composition within each pouch may be from about 300 mg to about 700 mg. If desired, other components can be contained within each pouch. For example, at least one flavored strip, piece or sheet of flavored water dispersible or water soluble material (e.g., a breath-freshening edible film type of material) may be disposed within each pouch along with or without at least one capsule. Such strips or sheets may be folded or crumpled in order to be readily incorporated within the pouch. See, for example, the types of materials and technologies set forth in U.S. Pat. No. 6,887,307 to Scott et al. and U.S. Pat. No. 6,923,981 to Leung et al.; and The EFSA Journal (2004) 85, 1-32; which are incorporated herein by reference.

### Active Ingredient

The composition as disclosed herein includes one or more active ingredients. As used herein, an “active ingredient” refers to one or more substances belonging to any of the following categories: API (active pharmaceutical ingredient), food additives, natural medicaments, and naturally occurring substances that can have an effect on humans. Example active ingredients include any ingredient known to impact one or more biological functions within the body, such as ingredients that furnish pharmacological activity or other direct effect in the diagnosis, cure, mitigation, treatment, or prevention of disease, or which affect the structure or any function of the body of humans (e.g., provide a stimulating action on the central nervous system, have an energizing effect, an antipyretic or analgesic action, or an otherwise useful effect on the body). In some embodiments, the active ingredient may be of the type generally referred to as dietary supplements, nutraceuticals, “phytochemicals” or “functional foods.” These types of additives are sometimes defined in the art as encompassing substances typically available from naturally-occurring sources (e.g., botanical materials) that provide one or more advantageous biological effects (e.g., health promotion, disease prevention, or other medicinal properties), but are not classified or regulated as drugs.

Non-limiting examples of active ingredients include those falling in the categories of botanical ingredients, stimulants, amino acids, nicotine components, and/or pharmaceutical, nutraceutical, and medicinal ingredients (e.g., vitamins, such as A, B3, B6, B12, and C, and/or cannabinoids, such as tetrahydrocannabinol (THC) and cannabidiol (CBD)). Each of these categories is further described herein below. The particular choice of active ingredients will vary depending upon the desired flavor, texture, and desired characteristics of the particular product.

In certain embodiments, the active ingredient is selected from the group consisting of caffeine, taurine, GABA, theanine, vitamin C, lemon balm extract, ginseng, citicoline, sunflower lecithin, and combinations thereof. For example, the active ingredient can include a combination of caffeine, theanine, and optionally ginseng. In another embodiment, the active ingredient includes a combination of theanine, gamma-amino butyric acid (GABA), and lemon balm extract. In a further embodiment, the active ingredient includes theanine, theanine and tryptophan, or theanine and one or more B vitamins (e.g., vitamin B6 or B12). In a still further embodiment, the active ingredient includes a combination of caffeine, taurine, and vitamin C.

The particular percentages of active ingredients present will vary depending upon the desired characteristics of the particular product. Typically, an active ingredient or combination thereof is present in a total concentration of at least about 0.001% by weight of the composition, such as in a range from about 0.001% to about 20%. In some embodiments, the active ingredient or combination of active ingredients is present in a concentration from about 0.1% w/w to about 10% by weight, such as, e.g., from about 0.5% w/w to about 10%, from about 1% to about 10%, from about 1% to about 5% by weight, based on the total weight of the composition. In some embodiments, the active ingredient or combination of active ingredients is present in a concentration of from about 0.001%, about 0.01%, about 0.1%, or about 1%, up to about 20% by weight, such as, e.g., from about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% by weight, based on the total weight of the composition. Further suitable ranges for specific active ingredients are provided herein below.

### Botanical

In some embodiments, the active ingredient comprises a botanical ingredient. As used herein, the term “botanical ingredient” or “botanical” refers to any plant material or fungal-derived material, including plant material in its natural form and plant material derived from natural plant materials, such as extracts or isolates from plant materials or treated plant materials (e.g., plant materials subjected to heat treatment, fermentation, bleaching, or other treatment processes capable of altering the physical and/or chemical nature of the material). For the purposes of the present disclosure, a “botanical” includes, but is not limited to, “herbal materials,” which refer to seed-producing plants that do not develop persistent woody tissue and are often valued for their medicinal or sensory characteristics (e.g., teas or tisanes). Reference to botanical material as “non-tobacco” is intended to exclude tobacco materials (i.e., does not include any Nicotiana species). In some embodiments, the compositions as disclosed herein can be characterized as free of any tobacco material (e.g., any embodiment as disclosed herein may be completely or substantially free of any tobacco material). By “substantially free” is meant that no tobacco material has been intentionally added. For example, certain embodiments can be characterized as having less than 0.001% by weight of tobacco, or less than 0.0001%, or even 0% by weight of tobacco.

When present, a botanical is typically at a concentration of from about 0.01% w/w to about 10% by weight, such as, e.g., from about 0.01% w/w, about 0.05%, about 0.1%, or about 0.5%, to about 1%, about 2%, about 3%, about 4%, about 5%, about 6%, about 7%, about 8%, about 9%, or about 10%, about 11%, about 12%, about 13%, about 14%, or about 15% by weight, based on the total weight of the composition.

The botanical materials useful in the present disclosure may comprise, without limitation, any of the compounds and sources set forth herein, including mixtures thereof. Certain botanical materials of this type are sometimes referred to as dietary supplements, nutraceuticals, “phytochemicals” or “functional foods.” Certain botanicals, as the plant material or an extract thereof, have found use in traditional herbal medicine, and are described further herein. Non-limiting examples of botanicals or botanical-derived materials include ashwagandha, Bacopa monniera, baobab, basil, Centella asiatica, Chai-hu, chamomile, cherry blossom, chlorophyll, cinnamon, citrus, cloves, cocoa, cordyceps, curcumin, damiana, Dorstenia arifolia, Dorstenia odorata, essential oils, eucalyptus, fennel, Galphimia glauca, ginger, Ginkgo biloba, ginseng (e.g., Panax ginseng), green tea, Griffonia simplicifolia, guarana, cannabis, hemp, hops, jasmine, Kaempferia parviflora (Thai ginseng), kava, lavender, lemon balm, lemongrass, licorice, lutein, maca, matcha, Nardostachys chinensis, oil-based extract of Viola odorata, peppermint, quercetin, resveratrol, Rhizoma gastrodiae, Rhodiola, rooibos, rose essential oil, rosemary, Sceletium tortuosum, Schisandra, Skullcap, spearmint extract, Spikenard, terpenes, tisanes, turmeric, Turnera aphrodisiaca, valerian, white mulberry, and Yerba mate.

In some embodiments, the active ingredient comprises lemon balm. Lemon balm (Melissa officinalis) is a mildly lemon-scented herb from the same family as mint (Lamiaceae). The herb is native to Europe, North Africa, and West Asia. The tea of lemon balm, as well as the essential oil and the extract, are used in traditional and alternative medicine. In some embodiments, the active ingredient comprises lemon balm extract. In some embodiments, the lemon balm extract is present in an amount of from about 1 to about 4% by weight, based on the total weight of the composition.

In some embodiments, the active ingredient comprises ginseng. Ginseng is the root of plants of the genus Panax, which are characterized by the presence of unique steroid saponin phytochemicals (ginsenosides) and gintonin. Ginseng finds use as a dietary supplement in energy drinks or herbal teas, and in traditional medicine. Cultivated species include Korean ginseng (P. ginseng), South China ginseng (P. notoginseng), and American ginseng (P. quinquefolius). American ginseng and Korean ginseng vary in the type and quantity of various ginsenosides present. In some embodiments, the ginseng is American ginseng or Korean ginseng. In specific embodiments, the active ingredient comprises Korean ginseng. In some embodiments, ginseng is present in an amount of from about 0.4 to about 0.6% by weight, based on the total weight of the composition.

### Stimulants

In some embodiments, the active ingredient comprises one or more stimulants. As used herein, the term “stimulant” refers to a material that increases activity of the central nervous system and/or the body, for example, enhancing focus, cognition, vigor, mood, alertness, and the like. Non-limiting examples of stimulants include caffeine, theacrine, theobromine, and theophylline. Theacrine (1,3,7,9-tetramethyluric acid) is a purine alkaloid which is structurally, related to caffeine, and possesses stimulant, analgesic, and anti-inflammatory effects. Present stimulants may be natural, naturally derived, or wholly synthetic. For example, certain botanical materials (guarana, tea, coffee, cocoa, and the like) may possess a stimulant effect by virtue of the presence of e.g., caffeine or related alkaloids, and accordingly are “natural” stimulants. By “naturally derived” is meant the stimulant (e.g., caffeine, theacrine) is in a purified form, outside its natural (e.g., botanical) matrix. For example, caffeine can be obtained by extraction and purification from botanical sources (e.g., tea). By “wholly synthetic”, it is meant that the stimulant has been obtained by chemical synthesis. In some embodiments, the active ingredient comprises caffeine. In some embodiments, the caffeine is present in an encapsulated form. On example of an encapsulated caffeine is Vitashure®, available from Balchem Corp., 52 Sunrise Park Road, New Hampton, N.Y., 10958.

When present, a stimulant or combination of stimulants (e.g., caffeine, theacrine, and combinations thereof) is typically at a concentration of from about 0.1% w/w to about 15% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, or about 15% by weight, based on the total weight of the composition. In some embodiments, the composition comprises caffeine in an amount of from about 1.5 to about 6% by weight, based on the total weight of the composition;

### Amino Acids

In some embodiments, the active ingredient comprises an amino acid. As used herein, the term “amino acid” refers to an organic compound that contains amine (—NH₂) and carboxyl (—COOH) or sulfonic acid (SO₃H) functional groups, along with a side chain (R group), which is specific to each amino acid. Amino acids may be proteinogenic or non-proteinogenic. By “proteinogenic” is meant that the amino acid is one of the twenty naturally occurring amino acids found in proteins. The proteinogenic amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. By “non-proteinogenic” is meant that either the amino acid is not found naturally in protein, or is not directly produced by cellular machinery (e.g., is the product of post-translational modification).

Non-limiting examples of non-proteinogenic amino acids include gamma-aminobutyric acid (GABA), taurine (2-aminoethanesulfonic acid), theanine (L-γ-glutamylethylamide), hydroxyproline, and beta-alanine. In some embodiments, the active ingredient comprises theanine. In some embodiments, the active ingredient comprises GABA. In some embodiments, the active ingredient comprises a combination of theanine and GABA. In some embodiments, the active ingredient is a combination of theanine, GABA, and lemon balm. In some embodiments, the active ingredient is a combination of caffeine, theanine, and ginseng. In some embodiments, the active ingredient comprises taurine. In some embodiments, the active ingredient is a combination of caffeine and taurine.

When present, an amino acid or combination of amino acids (e.g., theanine, GABA, and combinations thereof) is typically at a concentration of from about 0.1% w/w to about 15% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, or about 15% by weight, based on the total weight of the composition.

### Vitamins

In some embodiments, the active ingredient comprises a vitamin or combination of vitamins. As used herein, the term “vitamin” refers to an organic molecule (or related set of molecules) that is an essential micronutrient needed for the proper functioning of metabolism in a mammal. There are thirteen vitamins required by human metabolism, which are: vitamin A (as all-trans-retinol, all-trans-retinyl-esters, as well as all-trans-beta-carotene and other provitamin A carotenoids), vitamin B1 (thiamine), vitamin B2 (riboflavin), vitamin B3 (niacin), vitamin B5 (pantothenic acid), vitamin B6 (pyridoxine), vitamin B7 (biotin), vitamin B9 (folic acid or folate), vitamin B12 (cobalamins), vitamin C (ascorbic acid), vitamin D (calciferols), vitamin E (tocopherols and tocotrienols), and vitamin K (quinones). In some embodiments, the active ingredient comprises vitamin C. In some embodiments, the active ingredient is a combination of vitamin C, caffeine, and taurine.

When present, a vitamin or combination of vitamins (e.g., vitamin B6, vitamin B12, vitamin E, vitamin C, or a combination thereof) is typically at a concentration of from about 0.01% w/w to about 6% by weight, such as, e.g., from about 0.01%, about 0.02%, about 0.03%, about 0.04%, about 0.05%, about 0.06%, about 0.07%, about 0.08%, about 0.09%, or about 0.1% w/w, to about 0.2%, about 0.3%, about 0.4%, about 0.5% about 0.6%, about 0.7%, about 0.8%, about 0.9%, about 1%, about 2%, about 3%, about 4%, about 5%, or about 6% by weight, based on the total weight of the composition.

### Antioxidants

In some embodiments, the active ingredient comprises one or more antioxidants. As used herein, the term “antioxidant” refers to a substance which prevents or suppresses oxidation by terminating free radical reactions, and may delay or prevent some types of cellular damage. Antioxidants may be naturally occurring or synthetic. Naturally occurring antioxidants include those found in foods and botanical materials. Non-limiting examples of antioxidants include certain botanical materials, vitamins, polyphenols, and phenol derivatives.

Examples of botanical materials which are associated with antioxidant characteristics include without limitation acai berry, alfalfa, allspice, annatto seed, apricot oil, basil, bee balm, wild bergamot, black pepper, blueberries, borage seed oil, bugleweed, cacao, calamus root, catnip, catuaba, cayenne pepper, chaga mushroom, chervil, cinnamon, dark chocolate, potato peel, grape seed, ginseng, gingko biloba, Saint John's Wort, saw palmetto, green tea, black tea, black cohosh, cayenne, chamomile, cloves, cocoa powder, cranberry, dandelion, grapefruit, honeybush, echinacea, garlic, evening primrose, feverfew, ginger, goldenseal, hawthorn, hibiscus flower, jiaogulan, kava, lavender, licorice, marjoram, milk thistle, mints (menthe), oolong tea, beet root, orange, oregano, papaya, pennyroyal, peppermint, red clover, rooibos (red or green), rosehip, rosemary, sage, clary sage, savory, spearmint, spirulina, slippery elm bark, sorghum bran hi-tannin, sorghum grain hi-tannin, sumac bran, comfrey leaf and root, goji berries, gutu kola, thyme, turmeric, uva ursi, valerian, wild yam root, wintergreen, yacon root, yellow dock, yerba mate, yerba santa, bacopa monniera, withania somnifera, Lion's mane, and silybum marianum. Such botanical materials may be provided in fresh or dry form, essential oils, or may be in the form of an extracts. The botanical materials (as well as their extracts) often include compounds from various classes known to provide antioxidant effects, such as minerals, vitamins, isoflavones, phytoesterols, allyl sulfides, dithiolthiones, isothiocyanates, indoles, lignans, flavonoids, polyphenols, and carotenoids. Examples of compounds found in botanical extracts or oils include ascorbic acid, peanut endocarb, resveratrol, sulforaphane, beta-carotene, lycopene, lutein, co-enzyme Q, carnitine, quercetin, kaempferol, and the like. See, e.g., Santhosh et al., Phytomedicine, 12 (2005) 216-220, which is incorporated herein by reference.

Non-limiting examples of other suitable antioxidants include citric acid, Vitamin E or a derivative thereof, a tocopherol, epicatechol, epigallocatechol, epigallocatechol gallate, erythorbic acid, sodium erythorbate, 4-hexylresorcinol, theaflavin, theaflavin monogallate A or B, theaflavin digallate, phenolic acids, glycosides, quercitrin, isoquercitrin, hyperoside, polyphenols, catechols, resveratrols, oleuropein, butylated hydroxyanisole (BHA), butylated hydroxytoluene (BHT), tertiary butylhydroquinone (TBHQ), and combinations thereof.

When present, an antioxidant is typically at a concentration of from about 0.001% w/w to about 10% by weight, such as, e.g., from about 0.001%, about 0.005%, about 0.01% w/w, about 0.05%, about 0.1%, or about 0.5%, to about 1%, about 2%, about 3%, about 4%, about 5%, about 6%, about 7%, about 8%, about 9%, or about 10%, based on the total weight of the composition.

### Nicotine Component

In certain embodiments, the active ingredient comprises a nicotine component. By “nicotine component” is meant any suitable form of nicotine (e.g., free base or salt) for providing oral absorption of at least a portion of the nicotine present. Typically, the nicotine component is selected from the group consisting of nicotine free base and a nicotine salt. In some embodiments, the nicotine component is nicotine in its free base form, which easily can be adsorbed in for example, a microcrystalline cellulose material to form a microcrystalline cellulose-nicotine carrier complex. See, for example, the discussion of nicotine in free base form in US Pat. Pub. No. 2004/0191322 to Hansson, which is incorporated herein by reference. In some embodiments, at least a portion of the nicotine component can be employed in the form of a salt. Salts of nicotine can be provided using the types of ingredients and techniques set forth in U.S. Pat. No. 2,033,909 to Cox et al. and Perfetti, Beitrage Tabakforschung Int., 12: 43-54 (1983), which are incorporated herein by reference. Additionally, salts of nicotine are available from sources such as Pfaltz and Bauer, Inc. and K&K Laboratories, Division of ICN Biochemicals, Inc. Typically, the nicotine component is selected from the group consisting of nicotine free base, a nicotine salt such as hydrochloride, dihydrochloride, monotartrate, bitartrate, sulfate, salicylate, and nicotine zinc chloride.

In some embodiments, at least a portion of the nicotine can be in the form of a resin complex of nicotine, where nicotine is bound in an ion-exchange resin, such as nicotine polacrilex, which is nicotine bound to, for example, a polymethacrylic acid, such as Amberlite IRP64, Purolite C115HMR, or Doshion P551. See, for example, U.S. Pat. No. 3,901,248 to Lichtneckert et al., which is incorporated herein by reference. Another example is a nicotine-polyacrylic carbomer complex, such as with Carbopol 974P. In some embodiments, nicotine may be present in the form of a nicotine polyacrylic complex.

Typically, the nicotine component (calculated as the free base) when present, is in a concentration of at least about 0.001% by weight of the composition, such as in a range from about 0.001% to about 10%. In some embodiments, the nicotine component is present in a concentration from about 0.1% w/w to about 10% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, or about 10% by weight, calculated as the free base and based on the total weight of the composition. In some embodiments, the nicotine component is present in a concentration from about 0.1% w/w to about 3% by weight, such as, e.g., from about 0.1% w/w to about 2.5%, from about 0.1% to about 2.0%, from about 0.1% to about 1.5%, or from about 0.1% to about 1% by weight, calculated as the free base and based on the total weight of the composition.

In some embodiments, the products or compositions of the disclosure can be characterized as free of any nicotine component (e.g., any embodiment as disclosed herein may be completely or substantially free of any nicotine component). By “substantially free” is meant that no nicotine has been intentionally added, beyond trace amounts that may be naturally present in e.g., a botanical material. For example, certain embodiments can be characterized as having less than 0.001% by weight of nicotine, or less than 0.0001%, or even 0% by weight of nicotine, calculated as the free base.

In some embodiments, the active ingredient comprises a nicotine component (e.g., any product or composition of the disclosure, in addition to comprising any active ingredient or combination of active ingredients as disclosed herein, may further comprise a nicotine component).

### Cannabinoids

In some embodiments, the active ingredient comprises one or more cannabinoids. As used herein, the term “cannabinoid” refers to a class of diverse chemical compounds that acts on cannabinoid receptors, also known as the endocannabinoid system, in cells that alter neurotransmitter release in the brain. Ligands for these receptor proteins include the endocannabinoids produced naturally in the body by animals; phytocannabinoids, found in cannabis; and synthetic cannabinoids, manufactured artificially. Cannabinoids found in cannabis include, without limitation: cannabigerol (CBG), cannabichromene (CBC), cannabidiol (CBD), tetrahydrocannabinol (THC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabinolic acid (THCA), and tetrahydrocannabivarinic acid (THCV A). In certain embodiments, the cannabinoid is selected from tetrahydrocannabinol (THC), the primary psychoactive compound in cannabis, and cannabidiol (CBD) another major constituent of the plant, but which is devoid of psychoactivity. All of the above compounds can be used in the form of an isolate from plant material or synthetically derived.

Alternatively, the active ingredient can be a cannabimimetic, which is a class of compounds derived from plants other than cannabis that have biological effects on the endocannabinoid system similar to cannabinoids. Examples include yangonin, alpha-amyrin or beta-amyrin (also classified as terpenes), cyanidin, curcumin (tumeric), catechin, quercetin, salvinorin A, N-acylethanolamines, and N-alkylamide lipids.

When present, a cannabinoid (e.g., CBD) or cannabimimetic is typically in a concentration of at least about 0.1% by weight of the composition, such as in a range from about 0.1% to about 30%, such as, e.g., from about 0.1%, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 15%, about 20%, or about 30% by weight, based on the total weight of the composition.

### Terpenes

Active ingredients suitable for use in the present disclosure can also be classified as terpenes, many of which are associated with biological effects, such as calming effects. Terpenes are understood to have the general formula of (C₅H₈)ₙ and include monoterpenes, sesquiterpenes, and diterpenes. Terpenes can be acyclic, monocyclic or bicyclic in structure. Some terpenes provide an entourage effect when used in combination with cannabinoids or cannabimimetics. Examples include beta-caryophyllene, linalool, limonene, beta-citronellol, linalyl acetate, pinene (alpha or beta), geraniol, carvone, eucalyptol, menthone, iso-menthone, piperitone, myrcene, beta-bourbonene, and germacrene, which may be used singly or in combination.

### Pharmaceutical Ingredients

In some embodiments, the active ingredient comprises an active pharmaceutical ingredient (API). The API can be any known agent adapted for therapeutic, prophylactic, or diagnostic use. These can include, for example, synthetic organic compounds, proteins and peptides, polysaccharides and other sugars, lipids, phospholipids, inorganic compounds (e.g., magnesium, selenium, zinc, nitrate), neurotransmitters or precursors thereof (e.g., serotonin, 5-hydroxytryptophan, oxitriptan, acetylcholine, dopamine, melatonin), and nucleic acid sequences, having therapeutic, prophylactic, or diagnostic activity. Non-limiting examples of APIs include analgesics and antipyretics (e.g., acetylsalicylic acid, acetaminophen, 3-(4-isobutylphenyl)propanoic acid), phosphatidylserine, myoinositol, docosahexaenoic acid (DHA, Omega-3), arachidonic acid (AA, Omega-6), S-adenosylmethionine (SAM), beta-hydroxy-beta-methylbutyrate (HMB), citicoline (cytidine-5′-diphosphate-choline), and cotinine. In some embodiments, the active ingredient comprises citicoline. In some embodiments, the active ingredient is a combination of citicoline, caffeine, theanine, and ginseng. In some embodiments, the active ingredient comprises sunflower lecithin. In some embodiments, the active ingredient is a combination of sunflower lecithin, caffeine, theanine, and ginseng.

The amount of API may vary. For example, when present, an API is typically at a concentration of from about 0.001% w/w to about 10% by weight, such as, e.g., from about 0.01%, about 0.02%, about 0.03%, about 0.04%, about 0.05%, about 0.06%, about 0.07%, about 0.08%, about 0.09%, about 0.1% w/w, about 0.2%, about 0.3%, about 0.4%, about 0.5% about 0.6%, about 0.7%, about 0.8%, about 0.9%, or about 1%, to about 2%, about 3%, about 4%, about 5%, about 6%, about 7%, about 8%, about 9%, or about 10% by weight, based on the total weight of the composition.

In some embodiments, the composition is substantially free of any API. By “substantially free of any API” means that the composition does not contain, and specifically excludes, the presence of any API as defined herein, such as any Food and Drug Administration (FDA) approved therapeutic agent intended to treat any medical condition.

### Flavorant

In some embodiments, the composition comprises a flavorant. As used herein, a “flavorant” or “flavoring agent” is any flavorful or aromatic substance capable of altering the sensory characteristics associated with the composition and/or with an oral product incorporating such a composition. Examples of sensory characteristics that can be modified by the flavoring agent include taste, mouthfeel, moistness, coolness/heat, and/or fragrance/aroma. Flavoring agents may be natural or synthetic, and the character of the flavors imparted thereby may be described, without limitation, as fresh, sweet, herbal, confectionary, floral, fruity, or spicy. Specific types of flavors include, but are not limited to, vanilla, coffee, chocolate/cocoa, cream, mint, spearmint, menthol, peppermint, wintergreen, eucalyptus, lavender, cardamon, nutmeg, cinnamon, clove, cascarilla, sandalwood, honey, jasmine, ginger, anise, sage, licorice, lemon, orange, apple, peach, lime, cherry, strawberry, termpenes, trigeminal sensates, and any combinations thereof. See also, Leffingwell et al., Tobacco Flavoring for Smoking Products, R. J. Reynolds Tobacco Company (1972), which is incorporated herein by reference. Flavorings also may include components that are considered moistening, cooling or smoothening agents, such as eucalyptus. These flavors may be provided neat (i.e., alone) or in a composite, and may be employed as concentrates or flavor packages (e.g., spearmint and menthol, orange and cinnamon; lime, pineapple, and the like). Representative types of components also are set forth in U.S. Pat. No. 5,387,416 to White et al.; US Pat. App. Pub. No. 2005/0244521 to Strickland et al.; and PCT Application Pub. No. WO 05/041699 to Quinter et al., each of which is incorporated herein by reference. In some instances, the flavoring agent may be provided in a spray-dried form or a liquid form.

The flavoring agent may be a volatile flavor component. As used herein, “volatile” refers to a chemical substance that forms a vapor readily at ambient temperatures (i.e., a chemical substance that has a high vapor pressure at a given temperature relative to a nonvolatile substance). Typically, a volatile flavor component has a molecular weight below about 400 Da, and often include at least one carbon-carbon double bond, carbon-oxygen double bond, or both. In one embodiment, the at least one volatile flavor component comprises one or more alcohols, aldehydes, aromatic hydrocarbons, ketones, esters, terpenes, terpenoids, or a combination thereof. Non-limiting examples of aldehydes include vanillin, ethyl vanillin, p-anisaldehyde, hexanal, furfural, isovaleraldehyde, cuminaldehyde, benzaldehyde, and citronellal. Non-limiting examples of ketones include 1-hydroxy-2-propanone and 2-hydroxy-3-methyl-2-cyclopentenone-1-one. Non-limiting examples of esters include allyl hexanoate, ethyl heptanoate, ethyl hexanoate, isoamyl acetate, and 3-methylbutyl acetate. Non-limiting examples of terpenes include sabinene, limonene, gamma-terpinene, beta-farnesene, nerolidol, thujone, myrcene, geraniol, nerol, citronellol, linalool, and eucalyptol. In one embodiment, the at least one volatile flavor component comprises one or more of ethyl vanillin, cinnamaldehyde, sabinene, limonene, gamma-terpinene, beta-farnesene, or citral. In one embodiment, the at least one volatile flavor component comprises ethyl vanillin.

The amount of flavorant utilized in the composition can vary, but is typically up to about 10 weight percent, and certain embodiments are characterized by a flavoring agent content of at least about 0.1 weight percent, such as about 0.5 to about 10 weight percent, about 1 to about 6 weight percent, or about 2 to about 5 weight percent, based on the total weight of the composition.

### Additional Ingredients

Representative types of additional ingredients that may comprise the composition as disclosed herein, or may be useful for the manufacture of products pouched as disclosed herein include fillers, thickeners, film formers, binders, buffers and pH control agents, antiadherents, glidants, sweeteners, humectants, preservatives and antioxidants, surfactants, colorants, lubricants, and processing aids. In some embodiments, the composition comprises further components, such as fillers, binders, humectants, sweeteners, salts, buffering agents, tobacco materials, and the like. Examples of components suitable for use in the present compositions are described further herein below.

### Fillers

Compositions as described herein may include a filler or combination of fillers. Fillers may fulfill multiple functions, such as enhancing certain organoleptic properties such as texture and mouthfeel, enhancing cohesiveness or compressibility of the product, and the like, depending on the product. Generally, the filler is a porous particulate material and is cellulose-based. For example, fillers are any non-tobacco plant material or derivative thereof, including cellulose materials derived from such sources. Examples of cellulosic non-tobacco plant material include cereal grains (e.g., maize, oat, barley, rye, buckwheat, and the like), sugar beet (e.g., FIBREX® brand filler available from International Fiber Corporation), bran fiber, and mixtures thereof. Non-limiting examples of derivatives of non-tobacco plant material include starches (e.g., from potato, wheat, rice, corn), natural cellulose, and modified cellulosic materials. Additional examples of potential fillers include maltodextrin, dextrose, calcium carbonate, calcium phosphate, lactose, mannitol, xylitol, and sorbitol. Combinations of fillers can also be used.

“Starch” as used herein may refer to pure starch from any source, modified starch, or starch derivatives. Starch is present, typically in granular form, in almost all green plants and in various types of plant tissues and organs (e.g., seeds, leaves, rhizomes, roots, tubers, shoots, fruits, grains, and stems). Starch can vary in composition, as well as in granular shape and size. Often, starch from different sources has different chemical and physical characteristics. A specific starch can be selected for inclusion in the composition based on the ability of the starch material to impart a specific organoleptic property to composition. Starches derived from various sources can be used. For example, major sources of starch include cereal grains (e.g., rice, wheat, and maize) and root vegetables (e.g., potatoes and cassava). Other examples of sources of starch include acorns, arrowroot, arracacha, bananas, barley, beans (e.g., favas, lentils, mung beans, peas, chickpeas), breadfruit, buckwheat, canna, chestnuts, colacasia, katakuri, kudzu, malanga, millet, oats, oca, Polynesian arrowroot, sago, sorghum, sweet potato, quinoa, rye, tapioca, taro, tobacco, water chestnuts, and yams. Certain starches are modified starches. A modified starch has undergone one or more structural modifications, often designed to alter its high heat properties. Some starches have been developed by genetic modifications, and are considered to be “genetically modified” starches. Other starches are obtained and subsequently modified by chemical, enzymatic, or physical means. For example, modified starches can be starches that have been subjected to chemical reactions, such as esterification, etherification, oxidation, depolymerization (thinning) by acid catalysis or oxidation in the presence of base, bleaching, transglycosylation and depolymerization (e.g., dextrinization in the presence of a catalyst), cross-linking, acetylation, hydroxypropylation, and/or partial hydrolysis. Enzymatic treatment includes subjecting native starches to enzyme isolates or concentrates, microbial enzymes, and/or enzymes native to plant materials, e.g., amylase present in corn kernels to modify corn starch. Other starches are modified by heat treatments, such as pregelatinization, dextrinization, and/or cold water swelling processes. Certain modified starches include monostarch phosphate, distarch glycerol, distarch phosphate esterified with sodium trimetaphosphate, phosphate distarch phosphate, acetylated distarch phosphate, starch acetate esterified with acetic anhydride, starch acetate esterified with vinyl acetate, acetylated distarch adipate, acetylated distarch glycerol, hydroxypropyl starch, hydroxypropyl distarch glycerol, and starch sodium octenyl succinate.

In some embodiments, the filler is a cellulose material or cellulose derivative. One particularly suitable filler for use in the compositions described herein is microcrystalline cellulose (“MCC”). The MCC may be synthetic or semi-synthetic, or it may be obtained entirely from natural celluloses. The MCC may be selected from the group consisting of AVICEL® grades PH-100, PH-102, PH-103, PH-105, PH-112, PH-113, PH-200, PH-300, PH-302, VIVACEL® grades 101, 102, 12, 20 and EMOCEL® grades 50M and 90M, and the like, and mixtures thereof. In one embodiment, the composition comprises MCC as a filler.

When present, the amount of filler can vary, but is typically up to about 75 percent by weight of the composition, based on the total weight of the composition. A typical range of filler within the composition can be from about 10 to about 75 percent by total weight of the composition, for example, from about 10, about 15, about 20, about 25, or about 30, to about 35, about 40, about 45, or about 50 weight percent (e.g., about 20 to about 50 weight percent or about 25 to about 45 weight percent). In certain embodiments, the amount of filler is at least about 10 percent by weight, such as at least about 20 percent, or at least about 25 percent, or at least about 30 percent, or at least about 35 percent, or at least about 40 percent, based on the total weight of the composition. In some embodiments, the composition of the disclosure can be characterized as completely free or substantially free of filler. For example, in some embodiments, the traditional role of a filler (e.g., MCC) may be served by the nanocellulose (e.g., CNF, CNC, or bacterial cellulose).

### Binders

A binder (or combination of binders) may be employed in certain embodiments, in amounts sufficient to provide the desired physical attributes and physical integrity to the composition as described herein, and binders also often function as thickening or gelling agents. Typical binders can be organic or inorganic, or a combination thereof. Representative binders include cellulose derivatives, povidone, sodium alginate, starch-based binders, pectin, carrageenan, pullulan, zein, and the like, and combinations thereof. In some embodiments, the binder comprises pectin or carrageenan or combinations thereof. The amount of binder utilized in the composition can vary, but is typically up to about 30 weight percent, and certain embodiments are characterized by a binder content of at least about 0.1% by weight, such as about 1 to about 30% by weight, or about 5 to about 10% by weight, based on the total weight of the composition.

In one embodiment, the binder comprises a cellulose derivative. In certain embodiments, the cellulose derivative is a cellulose ether (including carboxyalkyl ethers), meaning a cellulose polymer with the hydrogen of one or more hydroxyl groups in the cellulose structure replaced with an alkyl, hydroxyalkyl, or aryl group. Non-limiting examples of such cellulose derivatives include methylcellulose, hydroxypropylcellulose (“HPC”), hydroxypropylmethylcellulose (“HPMC”), hydroxyethyl cellulose, and carboxymethylcellulose (“CMC”). In one embodiment, the cellulose derivative is one or more of methylcellulose, HPC, HPMC, hydroxyethyl cellulose, and CMC. In one embodiment, the cellulose derivative is HPC. In one embodiment, the cellulose derivative is a combination of HPC and HPMC. In some embodiments, the composition comprises from about 1 to about 10% of a cellulose derivative by weight, based on the total weight of the composition, with certain embodiments comprising about 1 to about 5% by weight of cellulose derivative, for example, from about 1%, about 2%, or about 3%, to about 4%, or about 5% by weight of the composition.

In certain embodiments, the binder includes a gum, for example, a natural gum. As used herein, a natural gum refers to polysaccharide materials of natural origin that have binding properties, and which are also useful as a thickening or gelling agents. Representative natural gums derived from plants, which are typically water soluble to some degree, include xanthan gum, guar gum, gum arabic, ghatti gum, gum tragacanth, karaya gum, locust bean gum, gellan gum, and combinations thereof. When present, natural gum binder materials are typically present in an amount of up to about 5% by weight, for example, from about 0.1, about 0.2, about 0.3, about 0.4, about 0.5, about 0.6, about 0.7, about 0.8, about 0.9, or about 1%, to about 2, about 3, about 4, or about 5% by weight, based on the total weight of the composition.

### Humectants

In certain embodiments, one or more humectants may be employed in the composition. Examples of humectants include, but are not limited to, glycerin, propylene glycol, and the like. Where included, the humectant is typically provided in an amount sufficient to provide desired moisture attributes and physical properties to the composition. Further, in some instances, the humectant may impart desirable flow characteristics to the composition for depositing in a mold, forming a foam or gel, or the like. When present, a humectant will typically make up about 5% or less of the weight of the composition (e.g., from about 0.5 to about 5% by weight). When present, a representative amount of humectant is about 0.1% to about 1% by weight, or about 1% to about 5% by weight, based on the total weight of the composition.

### Tobacco Material

In some embodiments, the composition of the present disclosure may include a tobacco material. The tobacco material can vary in species, type, and form. Generally, the tobacco material is obtained from for a harvested plant of the Nicotiana species. Example Nicotiana species include N. tabacum, N. rustica, N. alata, N. arentsii, N. excelsior, N. forgetiana, N. glauca, N. glutinosa, N. gossei, N. kawakamii, N. knightiana, N. langsdorffi, N. otophora, N. setchelli, N. sylvestris, N. tomentosa, N. tomentosiformis, N. undulata, N. x sanderae, N. africana, N. amplexicaulis, N. benavidesii, N. bonariensis, N. debneyi, N. longiflora, N. maritina, N. megalosiphon, N. occidentalis, N. paniculata, N. plumbaginifolia, N. raimondii, N. rosulata, N. simulans, N. stocktonii, N. suaveolens, N. umbratica, N. velutina, N. wigandioides, N. acaulis, N. acuminata, N. attenuata, N. benthamiana, N. cavicola, N. clevelandii, N. cordifolia, N. corymbosa, N. fragrans, N. goodspeedii, N. linearis, N. miersii, N. nudicaulis, N. obtusifolia, N. occidentalis subsp. Hersperis, N. pauciflora, N. petunioides, N. quadrivalvis, N. repanda, N. rotundifolia, N. solanifolia, and N. spegazzinii. Various representative other types of plants from the Nicotiana species are set forth in Goodspeed, The Genus Nicotiana, (Chonica Botanica) (1954); U.S. Pat. No. 4,660,577 to Sensabaugh, Jr. et al.; U.S. Pat. No. 5,387,416 to White et al., U.S. Pat. No. 7,025,066 to Lawson et al.; U.S. Pat. No. 7,798,153 to Lawrence, Jr. and U.S. Pat. No. 8,186,360 to Marshall et al.; each of which is incorporated herein by reference. Descriptions of various types of tobaccos, growing practices and harvesting practices are set forth in Tobacco Production, Chemistry and Technology, Davis et al. (Eds.) (1999), which is incorporated herein by reference.

Nicotiana species from which suitable tobacco materials can be obtained can be derived using genetic-modification or crossbreeding techniques (e.g., tobacco plants can be genetically engineered or crossbred to increase or decrease production of components, characteristics or attributes). See, for example, the types of genetic modifications of plants set forth in U.S. Pat. No. 5,539,093 to Fitzmaurice et al.; U.S. Pat. No. 5,668,295 to Wahab et al.; U.S. Pat. No. 5,705,624 to Fitzmaurice et al.; U.S. Pat. No. 5,844,119 to Weigl; U.S. Pat. No. 6,730,832 to Dominguez et al.; U.S. Pat. No. 7,173,170 to Liu et al.; U.S. Pat. No. 7,208,659 to Colliver et al. and U.S. Pat. No. 7,230,160 to Benning et al.; US Patent Appl. Pub. No. 2006/0236434 to Conkling et al.; and PCT WO2008/103935 to Nielsen et al. See, also, the types of tobaccos that are set forth in U.S. Pat. No. 4,660,577 to Sensabaugh, Jr. et al.; U.S. Pat. No. 5,387,416 to White et al.; and U.S. Pat. No. 6,730,832 to Dominguez et al., each of which is incorporated herein by reference.

The Nicotiana species can, in some embodiments, be selected for the content of various compounds that are present therein. For example, plants can be selected on the basis that those plants produce relatively high quantities of one or more of the compounds desired to be isolated therefrom. In certain embodiments, plants of the Nicotiana species (e.g., Galpao commun tobacco) are specifically grown for their abundance of leaf surface compounds. Tobacco plants can be grown in greenhouses, growth chambers, or outdoors in fields, or grown hydroponically.

Various parts or portions of the plant of the Nicotiana species can be included within a composition as disclosed herein, as disclosed herein. For example, virtually all of the plant (e.g., the whole plant) can be harvested, and employed as such. Alternatively, various parts or pieces of the plant can be harvested or separated for further use after harvest. For example, the flower, leaves, stem, stalk, roots, seeds, and various combinations thereof, can be isolated for further use or treatment. In some embodiments, the tobacco material comprises tobacco leaf (lamina). The composition as disclosed herein can include processed tobacco parts or pieces, cured and aged tobacco in essentially natural lamina and/or stem form, a tobacco extract, extracted tobacco pulp (e.g., using water as a solvent), or a mixture of the foregoing (e.g., a mixture that combines extracted tobacco pulp with granulated cured and aged natural tobacco lamina).

In certain embodiments, the tobacco material comprises solid tobacco material selected from the group consisting of lamina and stems. The tobacco that is used for the mixture most preferably includes tobacco lamina, or a tobacco lamina and stem mixture (of which at least a portion is smoke-treated). Portions of the tobaccos within the mixture may have processed forms, such as processed tobacco stems (e.g., cut-rolled stems, cut-rolled-expanded stems or cut-puffed stems), or volume expanded tobacco (e.g., puffed tobacco, such as dry ice expanded tobacco (DIET)). See, for example, the tobacco expansion processes set forth in U.S. Pat. No. 4,340,073 to de la Burde et al.; U.S. Pat. No. 5,259,403 to Guy et al.; and U.S. Pat. No. 5,908,032 to Poindexter, et al.; and U.S. Pat. No. 7,556,047 to Poindexter, et al., all of which are incorporated by reference. In addition, the composition optionally may incorporate tobacco that has been fermented. See, also, the types of tobacco processing techniques set forth in PCT Application Publication No. WO2005/063060 to Atchley et al., which is incorporated herein by reference.

Where used within a composition as disclosed herein, the tobacco material is typically used in a form that can be described as particulate (i.e., shredded, ground, granulated, or powder form). The tobacco plant or portion thereof can be separated into individual parts or pieces (e.g., the leaves can be removed from the stems, and/or the stems and leaves can be removed from the stalk). The harvested plant or individual parts or pieces can be further subdivided into parts or pieces (e.g., the leaves can be shredded, cut, comminuted, pulverized, milled or ground into pieces or parts that can be characterized as filler-type pieces, granules, particulates or fine powders).

The manner by which the tobacco material is provided in a finely divided or powder type of form may vary. Preferably, plant parts or pieces are comminuted, ground or pulverized into a particulate form using equipment and techniques for grinding, milling, or the like. Most preferably, the plant material is relatively dry in form during grinding or milling, using equipment such as hammer mills, cutter heads, air control mills, or the like. For example, tobacco parts or pieces may be ground or milled when the moisture content thereof is less than about 15 weight percent or less than about 5 weight percent. The plant, or parts thereof, can be subjected to external forces or pressure (e.g., by being pressed or subjected to roll treatment). When carrying out such processing conditions, the plant or portion thereof can have a moisture content that approximates its natural moisture content (e.g., its moisture content immediately upon harvest), a moisture content achieved by adding moisture to the plant or portion thereof, or a moisture content that results from the drying of the plant or portion thereof. For example, powdered, pulverized, ground or milled pieces of plants or portions thereof can have moisture contents of less than about 25 weight percent, often less than about 20 weight percent, and frequently less than about 15 weight percent. Most preferably, the tobacco material is employed in the form of parts or pieces that have an average particle size between 1.4 millimeters and 250 microns. In some instances, the tobacco particles may be sized to pass through a screen mesh to obtain the particle size range required. If desired, air classification equipment may be used to ensure that small sized tobacco particles of the desired sizes, or range of sizes, may be collected. If desired, differently sized pieces of granulated tobacco may be mixed together.

For the preparation of compositions, it is typical for a harvested plant of the Nicotiana species to be subjected to a curing process. The tobacco materials incorporated within the composition as disclosed herein are those that have been appropriately cured and/or aged. Descriptions of various types of curing processes for various types of tobaccos are set forth in Tobacco Production, Chemistry and Technology, Davis et al. (Eds.) (1999). Examples of techniques and conditions for curing flue-cured tobacco are set forth in Nestor et al., Beitrage Tabakforsch. Int., 20, 467-475 (2003) and U.S. Pat. No. 6,895,974 to Peele, which are incorporated herein by reference. Representative techniques and conditions for air curing tobacco are set forth in U.S. Pat. No. 7,650,892 to Groves et al.; Roton et al., Beitrage Tabakforsch. Int., 21, 305-320 (2005) and Staaf et al., Beitrage Tabakforsch. Int., 21, 321-330 (2005), which are incorporated herein by reference. Certain types of tobaccos can be subjected to alternative types of curing processes, such as fire curing or sun curing.

In certain embodiments, tobacco materials that can be employed include flue-cured or Virginia (e.g., K326), burley, sun-cured (e.g., Indian Kurnool and Oriental tobaccos, including Katerini, Prelip, Komotini, Xanthi and Yambol tobaccos), Maryland, dark, dark-fired, dark air cured (e.g., Madole, Passanda, Cubano, Jatin and Bezuki tobaccos), light air cured (e.g., North Wisconsin and Galpao tobaccos), Indian air cured, Red Russian and Rustica tobaccos, as well as various other rare or specialty tobaccos and various blends of any of the foregoing tobaccos.

The tobacco material may also have a so-called “blended” form. For example, the tobacco material may include a mixture of parts or pieces of flue-cured, burley (e.g., Malawi burley tobacco) and Oriental tobaccos (e.g., as tobacco composed of, or derived from, tobacco lamina, or a mixture of tobacco lamina and tobacco stem). For example, a representative blend may incorporate about 30 to about 70 parts burley tobacco (e.g., lamina, or lamina and stem), and about 30 to about 70 parts flue cured tobacco (e.g., stem, lamina, or lamina and stem) on a dry weight basis. Other example tobacco blends incorporate about 75 parts flue-cured tobacco, about 15 parts burley tobacco, and about 10 parts Oriental tobacco; or about 65 parts flue-cured tobacco, about 25 parts burley tobacco, and about 10 parts Oriental tobacco; or about 65 parts flue-cured tobacco, about 10 parts burley tobacco, and about 25 parts Oriental tobacco; on a dry weight basis. Other example tobacco blends incorporate about 20 to about 30 parts Oriental tobacco and about 70 to about 80 parts flue-cured tobacco on a dry weight basis.

Tobacco materials used in the present disclosure can be subjected to, for example, fermentation, bleaching, and the like. If desired, the tobacco materials can be, for example, irradiated, pasteurized, or otherwise subjected to controlled heat treatment. Such treatment processes are detailed, for example, in U.S. Pat. No. 8,061,362 to Mua et al., which is incorporated herein by reference. In certain embodiments, tobacco materials can be treated with water and an additive capable of inhibiting reaction of asparagine to form acrylamide upon heating of the tobacco material (e.g., an additive selected from the group consisting of lysine, glycine, histidine, alanine, methionine, cysteine, glutamic acid, aspartic acid, proline, phenylalanine, valine, arginine, compositions incorporating di- and trivalent cations, asparaginase, certain non-reducing saccharides, certain reducing agents, phenolic compounds, certain compounds having at least one free thiol group or functionality, oxidizing agents, oxidation catalysts, natural plant extracts (e.g., rosemary extract), and combinations thereof). See, for example, the types of treatment processes described in U.S. Pat. Nos. 8,434,496, 8,944,072, and 8,991,403 to Chen et al., which are all incorporated herein by reference. In certain embodiments, this type of treatment is useful where the original tobacco material is subjected to heat in the processes previously described.

In various embodiments, the tobacco material can be treated to extract a soluble component of the tobacco material therefrom. “Tobacco extract” as used herein refers to the isolated components of a tobacco material that are extracted from solid tobacco pulp by a solvent that is brought into contact with the tobacco material in an extraction process. Various extraction techniques of tobacco materials can be used to provide a tobacco extract and tobacco solid material. See, for example, the extraction processes described in US Pat. Appl. Pub. No. 2011/0247640 to Beeson et al., which is incorporated herein by reference. Other example techniques for extracting components of tobacco are described in U.S. Pat. No. 4,144,895 to Fiore; U.S. Pat. No. 4,150,677 to Osborne, Jr. et al.; U.S. Pat. No. 4,267,847 to Reid; U.S. Pat. No. 4,289,147 to Wildman et al.; U.S. Pat. No. 4,351,346 to Brummer et al.; U.S. Pat. No. 4,359,059 to Brummer et al.; U.S. Pat. No. 4,506,682 to Muller; U.S. Pat. No. 4,589,428 to Keritsis; U.S. Pat. No. 4,605,016 to Soga et al.; U.S. Pat. No. 4,716,911 to Poulose et al.; U.S. Pat. No. 4,727,889 to Niven, Jr. et al.; U.S. Pat. No. 4,887,618 to Bernasek et al.; U.S. Pat. No. 4,941,484 to Clapp et al.; U.S. Pat. No. 4,967,771 to Fagg et al.; U.S. Pat. No. 4,986,286 to Roberts et al.; U.S. Pat. No. 5,005,593 to Fagg et al.; U.S. Pat. No. 5,018,540 to Grubbs et al.; U.S. Pat. No. 5,060,669 to White et al.; U.S. Pat. No. 5,065,775 to Fagg; U.S. Pat. No. 5,074,319 to White et al.; U.S. Pat. No. 5,099,862 to White et al.; U.S. Pat. No. 5,121,757 to White et al.; U.S. Pat. No. 5,131,414 to Fagg; U.S. Pat. No. 5,131,415 to Munoz et al.; U.S. Pat. No. 5,148,819 to Fagg; U.S. Pat. No. 5,197,494 to Kramer; U.S. Pat. No. 5,230,354 to Smith et al.; U.S. Pat. No. 5,234,008 to Fagg; U.S. Pat. No. 5,243,999 to Smith; U.S. Pat. No. 5,301,694 to Raymond et al.; U.S. Pat. No. 5,318,050 to Gonzalez-Parra et al.; U.S. Pat. No. 5,343,879 to Teague; U.S. Pat. No. 5,360,022 to Newton; U.S. Pat. No. 5,435,325 to Clapp et al.; U.S. Pat. No. 5,445,169 to Brinkley et al.; U.S. Pat. No. 6,131,584 to Lauterbach; U.S. Pat. No. 6,298,859 to Kierulff et al.; U.S. Pat. No. 6,772,767 to Mua et al.; and U.S. Pat. No. 7,337,782 to Thompson, all of which are incorporated by reference herein.

In some embodiments, the type of tobacco material is selected such that it is initially visually lighter in color than other tobacco materials to some degree (e.g., whitened or bleached). Tobacco pulp can be whitened in certain embodiments according to any means known in the art.

Typical inclusion ranges for tobacco materials can vary depending on the nature and type of the tobacco material, and the intended effect on the composition, with an example range of up to about 30% by weight (or up to about 20% by weight or up to about 10% by weight or up to about 5% by weight), based on total weight of the composition (e.g., about 0.1 to about 15% by weight). In some embodiments, the composition can be characterized as completely free or substantially free of tobacco material (other than purified nicotine as a possible active ingredient). In some embodiments, such products are described as having no tobacco material (other than purified nicotine as a possible active ingredient) intentionally added thereto. For example, certain embodiments can be characterized as having less than 1% by weight, or less than 0.5% by weight, or less than 0.1% by weight of tobacco material, or 0% by weight of tobacco material based on the weight of the composition.

### Salts

In some embodiments, the composition within pouched products according to the disclosure comprises a salt (e.g., an alkali metal salt), typically employed in an amount sufficient to provide desired sensory attributes to the composition. Non-limiting examples of suitable salts include sodium chloride, potassium chloride, ammonium chloride, flour salt, sodium acetate, sodium citrate, and the like. When present, a representative amount of salt is about 0.5 percent by weight or more, about 1.0 percent by weight or more, or about 1.5 percent by weight or more, but will typically make up about 10 percent or less, or about 7.5 percent or less, or about 5 percent or less (e.g., from about 0.5 to about 5 percent by weight) of the total weight of the composition

### Sweeteners

In order to improve the sensory properties of the composition within pouched products according to the disclosure, one or more sweeteners may be added. The sweeteners can be any sweetener or combination of sweeteners, in natural or artificial form, or as a combination of natural and artificial sweeteners. Examples of natural sweeteners include fructose, sucrose, glucose, maltose, mannose, galactose, lactose, isomaltulose, stevia, honey, and the like. Examples of artificial sweeteners include sucralose, maltodextrin, saccharin, aspartame, acesulfame K, neotame and the like. In some embodiments, the sweetener comprises one or more sugar alcohols. Sugar alcohols are polyols derived from monosaccharides or disaccharides that have a partially or fully hydrogenated form. Sugar alcohols have, for example, about 4 to about 20 carbon atoms and include erythritol, arabitol, ribitol, isomalt, maltitol, dulcitol, iditol, mannitol, xylitol, lactitol, sorbitol, and combinations thereof (e.g., hydrogenated starch hydrolysates).

When present, a sweetener or combination of sweeteners may make up from about 0.1 to about 20 percent or more by weight of the of the composition, for example, from about 0.1 to about 1%, from about 1 to about 5%, from about 5 to about 10%, or from about 10 to about 20% by weight, based on the total weight of the composition.

### Buffering Agents

In certain embodiments, the composition within the pouched products of the present disclosure can comprise pH adjusters or buffering agents. Examples of pH adjusters and buffering agents that can be used include, but are not limited to, metal hydroxides (e.g., alkali metal hydroxides such as sodium hydroxide and potassium hydroxide), and other alkali metal buffers such as metal carbonates (e.g., potassium carbonate or sodium carbonate), or metal bicarbonates such as sodium bicarbonate, and the like. Where present, the buffering agent is typically present in an amount less than about 5 percent based on the weight of the composition, for example, from about 0.5% to about 5%, such as, e.g., from about 0.75% to about 4%, from about 0.75% to about 3%, or from about 1% to about 2% by weight, based on the total weight of the composition. Non-limiting examples of suitable buffers include alkali metals acetates, glycinates, phosphates, glycerophosphates, citrates, carbonates, hydrogen carbonates, borates, or mixtures thereof.

### Colorants

A colorant may be employed in amounts sufficient to provide the desired physical attributes to the composition within the pouched products according to the present disclosure. Examples of colorants include various dyes and pigments, such as caramel coloring and titanium dioxide. The amount of colorant utilized in the composition can vary, but when present is typically up to about 3 weight percent, such as from about 0.1%, about 0.5%, or about 1%, to about 3% by weight, based on the total weight of the composition.

### Oral Care Ingredients

Oral care ingredients provide the ability to inhibit tooth decay or loss, inhibit gum disease, relieve mouth pain, whiten teeth, or otherwise inhibit tooth staining, elicit salivary stimulation, inhibit breath malodor, freshen breath, or the like. For example, effective amounts of ingredients such as thyme oil, eucalyptus oil and zinc (e.g., such as the ingredients of formulations commercially available as ZYTEX® from Discus Dental) can be incorporated into the composition as disclosed herein. Other examples of ingredients that can be incorporated in desired effective amounts within the present composition can include those that are incorporated within the types of oral care compositions set forth in Takahashi et al., Oral Microbiology and Immunology, 19(1), 61-64 (2004); U.S. Pat. No. 6,083,527 to Thistle; and US Pat. Appl. Pub. Nos. 2006/0210488 to Jakubowski and 2006/02228308 to Cummins et al. Other exemplary ingredients include those contained in formulations marketed as MALTISORB® by Roquette and DENTIZYME® by NatraRx. When present, a representative amount of oral care additive is at least about 1 percent, often at least about 3 percent, and frequently at least about 5 percent of the total weight of the composition. The amount of oral care additive will not typically exceed about 30 percent, often will not exceed about 25 percent, and frequently will not exceed about 20 percent, of the total weight of the composition.

### Other Additives

Other additives can be included in the composition as disclosed. For example, the composition can be processed, blended, formulated, combined, and/or mixed with other materials or ingredients. The additives can be artificial, or can be obtained or derived from herbal or biological sources. Examples of further types of additives include thickening or gelling agents (e.g., fish gelatin), preservatives (e.g., potassium sorbate and the like), disintegration aids, zinc or magnesium salts selected to be relatively water-soluble for compositions with greater water solubility (e.g., magnesium or zinc gluconate) or selected to be relatively water-insoluble for compositions with reduced water solubility (e.g., magnesium or zinc oxide), or combinations thereof. See, for example, those representative components, combination of components, relative amounts of those components, and manners and methods for employing those components, set forth in U.S. Pat. No. 9,237,769 to Mua et al., U.S. Pat. No. 7,861,728 to Holton, Jr. et al., US Pat. App. Pub. No. 2010/0291245 to Gao et al., and US Pat. App. Pub. No. 2007/0062549 to Holton, Jr. et al., each of which is incorporated herein by reference. Typical inclusion ranges for such additional additives can vary depending on the nature and function of the additive and the intended effect on the final composition, with an example range of up to about 10% by weight, (e.g., about 0.1 to about 5% by weight) based on total weight of the composition.

The aforementioned additives can be employed together (e.g., as additive formulations) or separately (e.g., individual additive components can be added at different stages involved in the preparation of the final product). Furthermore, the aforementioned types of additives may be encapsulated as provided in the final product or composition. Exemplary encapsulated additives are described, for example, in WO2010/132444 to Atchley, which is incorporated by reference herein.

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1. An oral pouched product in the form of a porous pouch defining a cavity containing a composition adapted for oral use, wherein the porous pouch is formed from a fleece material comprising two or more layers, and wherein the two or more layers are in direct contact with one another.

2. The oral pouched product of claim 1, wherein the two or more layers are laminated to one another.

3. The oral pouched product of claim 1, wherein the fleece material comprises three or more layers.

4. The oral pouched product of claim 1, wherein the two or more layers comprise an inner layer adjacent to the composition adapted for oral use and an outer layer forming an outer surface of the pouched product, wherein the inner layer and the outer layer each have different physical properties.

5. The oral pouched product of claim 4, wherein the physical properties are selected from the group consisting of softness, mouth feel, dissolution properties, stain resistance, and combinations thereof.

6. The oral pouched product of claim 4, wherein the outer layer is softer than the inner layer.

7. The oral pouched product of claim 4, wherein at least one of the inner layer and the outer layer comprises an active ingredient, a flavorant, or both.

8. The oral pouched product of claim 7, wherein the active ingredient is selected from the group consisting of a botanical material, a stimulant, an amino acid, a vitamin, an antioxidant, nicotine components, a nutraceutical, a cannabinoid, a cannabimimetic, a terpene, a pharmaceutical agent, and combinations thereof.

9. The oral pouched product of claim 7, wherein the outer layer comprises an active ingredient, a flavorant, or both; and wherein the outer layer is configured to provide fast release of the active ingredient, the flavorant, or both, and the inner layer is configured for stain resistance.

10. The oral pouched product of claim 7, wherein the outer layer comprises a first active ingredient, a first flavorant, or both; wherein the inner layer comprises a second active ingredient, a second flavorant, or both; and wherein the first active ingredient and the first flavorant are different from the second active ingredient and the second flavorant.

11. The oral pouched product of claim 2, wherein the laminated structure is prepared using needle punching, and the laminated structure is subsequently extruded.

12. The oral pouched product of claim 11, wherein two or more active ingredients are layered on the laminated structure prior to needle punching, and wherein the two or more active ingredients are distributed throughout the resulting pouched product.

13. A method of preparing the oral pouched product of claim 1, comprising: combining the two or more layers to give the fleece material; forming the porous pouch therefrom; and enclosing the composition adapted for oral use within the porous pouch.

14. An oral pouched product comprising a composition adapted for oral use within a porous pouch, the porous pouch comprising at least one layer of fleece material and at least one layer of a gel; wherein the gel is in direct contact with the at least one layer of fleece material.

15. The oral pouched product of claim 14, wherein the porous pouch comprises an inner surface and an outer surface, and wherein the at least one layer of gel is adhered to the inner surface of the porous pouch.

16. The oral pouched product of claim 14, wherein the porous pouch comprises an inner surface and an outer surface, and wherein the at least one layer of gel is adhered to the outer surface of the porous pouch.

17. The oral pouched product of claim 1, wherein the gel comprises an active ingredient, a flavorant, or both, and wherein the gel is configured for rapid release or gradual release of the active ingredient, the flavorant, or both.

18. The oral pouched product of claim 1, wherein the gel comprises one or more flavorants, providing for substantially immediate release of flavor when the pouched product is placed in a user's mouth.

19. A method of providing an oral product with a customized flavor release profile, comprising:
selecting a first nonwoven material based on its release or barrier properties;
selecting a second nonwoven material based on its release or barrier properties;
incorporating an active ingredient and/or flavorant within one or both of the first and second nonwoven materials;
constructing a multi-layered fleece material from the first and second nonwoven materials;
forming a porous pouch from the multi-layered fleece material; and
enclosing a composition adapted for oral use within the porous pouch.

20. The method of claim 19, wherein the release properties are correlated with one or more of composition, basis weight, thickness, porosity, and pore size.

21. A method of preventing discoloration of an oral pouched product, comprising:
selecting a first nonwoven material suitable to prevent passage of color associated with a composition adapted for oral use;
selecting a second nonwoven material;
constructing a multi-layered fleece material from the first and second nonwoven materials;
forming a porous pouch from the multi-layered fleece material and enclosing the composition adapted for oral use within the porous pouch,
such that the first nonwoven material is in direct contact with the composition adapted for oral use.
